SEQUENCE LISTING (22 sequences)

SEQ ID NO: 1 - DNA, 1177 bp, Artificial Sequence
  Description of Artificial Sequence/Note = synthetic construct
cagaccccttatatatggttctccaagccttcctcacctgcacactctttttacttttgaaatcatttccacagtggagacaggtgcttcctacccaccagaagaccccctaccagtgaatgttgacggagcttgcccaacttttcgtgcagtaagaagaaccagccaccttcacacagcagcctccagcgtcacttcaggacctgagcaggagcacgggccctttcttcaatgcagacaagcttgagacaacagattctccccggcctgagcctaatccttctcgttttgaaccaagtaccagagcttcagggtcaagagttccgatttgggccttgccaagtgaccggggtggttctcccagaactgtgggaggccttctggactgtgaagaacactgtgaaaactcaggacgagctcacaagtgtccggctgttgaaaccacaggttctgcagaatgtctcggatgccgagagctgttaccttgcccacagcctgctgaagttctacttgaacactgttttcaaaaactatcacagcaaaatagtcaaattcaaggtcttgaagtcattctccactctggccaacaactttttagtcatcatgtccaaactgcagcctagtaaggacaatgccatgcttcccattagtgacagtgcacgccggcgttttttgctgtaccacagaacattcaaacagttggacatagaagtggctttggcgaaagcctttggggaagtggacattctcctggcctggatgcagaatttctaccagctctgattgccaatccggataacttcctcctttgttctccgtgccatttcaaggcattgttcatatccctgttgtcctcagggcacttcagacccttggccatggacccctgtcgttggctcaggcttttcttcagacctcactctttagtccaaacgacagccatggacagcacctttggatgctccgactgacccacaacgtggatttgcatatttattacagccctatttaactaatgtcactgtttcggtagaaaccgtatttatttgtgagactggacgttccatgaaagcatcatgccccgtgtttgcaccttacttcctgtgagctggctcaccatgggggcagtagatggttgctcagtaaatatttaaaatggaaaaaaaaaaaaaaaaaa

SEQ ID NO: 2 - protein, 183 aa, Artificial Sequence
  Description of Artificial Sequence/Note = synthetic construct
MQTSLRQQILPGLSLILLVLNQVPELQGQEFRFGPCQVTGVVLPELWEAFWTVKNTVKTQDELTSVRLLKPQVLQNVSDAESCYLAHSLLKFYLNTVFKNYHSKIVKFKVLKSFSTLANNFLVIMSKLQPSKDNAMLPISDSARRRFLLYHRTFKQLDIEVALAKAFGEVDILLAWMQNFYQL

SEQ ID NO: 3 - DNA, 510 bp, Artificial Sequence
  Description of Artificial Sequence/Note = synthetic construct
tgaggctgcttgggaggaaggccaggaggaacacgagactgagagatgaattttcaacagaggctgcaaagcctgtggactttagccagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctgaatgtc

SEQ ID NO: 4 - protein, 154 aa, Artificial Sequence
  Description of Artificial Sequence/Note = synthetic construct
MNFQQRLQSLWTLASRPFCPPLLATASQMQMVVLPCLGFTLLLWSQVSGAQGQEFHFGPCQVKGVVPQKLWEAFWAVKDTMQAQDNITSARLLQQEVLQNVSQENEMFSIRDSAHRRFLLFRRAFKQLDVEAALTKALGEVDILLTWMQKFYKL

SEQ ID NO: 5 - DNA, 674 bp, Artificial Sequence
  Description of Artificial Sequence/Note = synthetic construct
tgaggctgcttgggaggaaggccaggaggaacacgagactgagagatgaattttcaacagaggctgcaaagcctgtggactttagccagcagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctctgaatgtc

SEQ ID NO: 6 - protein, 207 aa, Artificial Sequence
  Description of Artificial Sequence/Note = synthetic construct
MNFQQRLQSLWTLASRPFCPPLLATASQMQMVVLPCLGFTLLLWSQVSGAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKL

SEQ ID NO: 7 - DNA, 2051 bp, Artificial Sequence
  Description of Artificial Sequence/Note = synthetic construct
ggatcctcctctagactgccatgcagacaagcttgagacaacagattctccccggcctgagcctaatccttctcgttttgaaccaagtaccagagcttcagggtcaagagttccgatttgggccttgccaagtgaccggggtggttctcccagaactgtgggaggccttctggactgtgaagaacactgtgaaaactcaggacgagctcacaagtgtccggctgttgaaaccacaggttctgcagaatgtctcggatgccgagagctgttaccttgcccacagcctgctgaagttctacttgaacactgttttcaaaaactatcacagcaaaatagtcaaattcaaggtcttgaagtcattctccactctggccaacaactttttagtcatcatgtccaaactgcagcctagtaaggacaatgccatgcttcccattagtgacagtgcacgccggcgttttttgctgtaccacagaacattcaaacagttggacatagaagtggctttggcgaaagcctttggggaagtggacattctcctggcctggatgcagaatttctaccagctcggatcttccggaatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcccatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttaa

SEQ ID NO: 8 - protein, 676 aa, Artificial Sequence
  Description of Artificial Sequence/Note = synthetic construct
MQTSLRQQILPGLSLILLVLNQVPELQGQEFRFGPCQVTGVVLPELWEAFWTVKNTVKTQDELTSVRLLKPQVLQNVSDAESCYLAHSLLKFYLNTVFKNYHSKIVKFKVLKSFSTLANNFLVIMSKLQPSKDNAMLPISDSARRRFLLYHRTFKQLDIEVALAKAFGEVDILLAWMQNFYQLGSSGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFPMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPG

SEQ ID NO: 9 - DNA, 2121 bp, Artificial Sequence
  Description of Artificial Sequence/Note = synthetic construct
ggatcccgagactgagagatgaattttcaacagaggctgcaaagcctgtggactttagccagcagacccttctgccctcctttgctggcgacagcctctcaaatgcagatggttgtgctcccttgcctgggttttaccctgcttctctggagccaggtatcaggggcccagggccaagaattccactttgggccctgccaagtgaagggggttgttccccagaaactgtgggaagccttctgggctgtgaaagacactatgcaagctcaggataacatcacgagtgcccggctgctgcagcaggaggttctgcagaacgtctcggatgctgagagctgttaccttgtccacaccctgctggagttctacttgaaaactgttttcaaaaactaccacaatagaacagttgaagtcaggactctgaagtcattctctactctggccaacaactttgttctcatcgtgtcacaactgcaacccagtcaagaaaatgagatgttttccatcagagacagtgcacacaggcggtttctgctattccggagagcattcaaacagttggacgtagaagcagctctgaccaaagcccttggggaagtggacattcttctgacctggatgcagaaattctacaagctcggatcttccggaatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcccatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttaa

SEQ ID NO: 10 - protein, 700 aa, Artificial Sequence
  Description of Artificial Sequence/Note = synthetic construct
MNFQQRLQSLWTLASRPFCPPLLATASQMQMVVLPCLGFTLLLWSQVSGAQGQEFHFGPCQVKGVVPQKLWEAFWAVKDTMQAQDNITSARLLQQEVLQNVSDAESCYLVHTLLEFYLKTVFKNYHNRTVEVRTLKSFSTLANNFVLIVSQLQPSQENEMFSIRDSAHRRFLLFRRAFKQLDVEAALTKALGEVDILLTWMQKFYKLGSSGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFPMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPG

SEQ ID NO: 11 - DNA, 17 bp, Artificial Sequence
  Description of Artificial Sequence/Note = synthetic construct
aagatctgaggctgctt

SEQ ID NO: 12 - DNA, 27 bp, Artificial Sequence
  Description of Artificial Sequence/Note = synthetic construct
ccagatctagacattcagagcttgtag

SEQ ID NO: 13 - DNA, 16 bp, Artificial Sequence
  Description of Artificial Sequence/Note = synthetic construct
ccagagcttcagggtc

SEQ ID NO: 14 - DNA, 20 bp, Artificial Sequence
  Description of Artificial Sequence/Note = synthetic construct
aagctttccggattggcaat

SEQ ID NO: 15 - DNA, 18 bp, Artificial Sequence
  misc_feature (0)...(0): Note  B can be G or T(U) or C
ggatccabbtatcagggg

SEQ ID NO: 16 - DNA, 22 bp, Artificial Sequence
  Description of Artificial Sequence/Note = synthetic construct
aagcttgtagaatttctgcatc

SEQ ID NO: 17 - DNA, 25 bp, Artificial Sequence
  Description of Artificial Sequence/Note = synthetic construct
tgcaaagcctgtggactttagccag

SEQ ID NO: 18 - DNA, 25 bp, Artificial Sequence
  Description of Artificial Sequence/Note = synthetic construct
ccgcctgtgtgcactgtctctgatg

SEQ ID NO: 19 - DNA, 27 bp, Artificial Sequence
  Description of Artificial Sequence/Note = synthetic construct
ccaggaggaacacgagactgagagatg

SEQ ID NO: 20 - DNA, 22 bp, Artificial Sequence
  Description of Artificial Sequence/Note = synthetic construct
ggaggtccaggtctagacattc

SEQ ID NO: 21 - DNA, 25 bp, Artificial Sequence
  Description of Artificial Sequence/Note = synthetic construct
tgcaaagcctgtggactttagccag

SEQ ID NO: 22 - DNA, 25 bp, Artificial Sequence
  Description of Artificial Sequence/Note = synthetic construct
gaaaacatctcattttcttcgagac

(19) United States
(12) Patent Application Publication
(10) Pub. No.: US 2004/0019193 A1
(43) Pub. Date: Jan. 29, 2004

(54) Mob-5/hmob-5 as a cancer diagnostic marker

(76) Inventor: Peng Liang, Nashville, TN (US)

Correspondence Address: NEEDLE & ROSENBERG, P.C., SUITE 1000, 999 PEACHTREE STREET, ATLANTA, GA, 30309-3915, US

(21) Appl. No.: 10/182,094

(22) PCT Filed: Jan. 26, 2001
(86) PCT No.: PCT/US01/02680

(51) Int. Cl. C12Q 1/68; G01N 33/574; C07H 21/04; C12P 21/02; C12N 5/06; C07K 14/45; C07K 16/30

(52) U.S. Cl. 536/23.5; 435/6; 435/7.23; 435/69.3; 435/320.1; 435/325; 530/350; 530/388.8

(57) ABSTRACT

The invention provides isolated nucleic acids comprising the nucleic acids set forth in the Sequence Listing as SEQ ID NO: 1(corresponding to a cDNA encoding a rat Mob-5 protein), SEQ ID NO: 3(corresponding to a cDNA encoding the cancer specific human Mob-5 protein homolog with an internal deletion of 53 amino acid residues, referred to as cMob-5.), SEQ ID NO: 5(corresponding to a cDNA encoding a human Mob-5 protein), SEQ ID NO: 7(corresponding to a nucleic acid encoding a rat Mob-5-AP fusion protein and SEQ ID NO: 9(corresponding to a nucleic acid encoding a human Mob-5-AP fusion protein). The invention also provides purified polypeptides having the sequences set forth in the Sequence Listing as any of SEQ ID NO: 2, SEQ ID NO: 4, SEQ ID NO: 6, SEQ ID NO: 8 and SEQ ID NO: 10. This invention also relates to a method of detecting the presence of cancer in a patient comprising: a) contacting a sample from the patient with an antibody to Mob-5; and b) detecting the binding of the antibody with an antigen in the sample, wherein binding of antigen to the antibody indicates the presence of Mob-5 antigen in the sample and wherein Mob-5 antigen in the sample indicates the presence of cancer in the patient, thereby detecting the presence of cancer in the patient.

This application claims priority to U.S. provisional application Serial No. 60/178,185 filed on Jan. 26, 2000. The 60/178,185 provisional patent application is herein incorporated by this reference in its entirety.

This invention was made with government support under Grant CA74067 from the National Institutes of Health. The U.S. Government may have certain rights in this invention.

### BACKGROUND OF THE INVENTION

1. Field of the Invention

This invention relates to the discovery of a novel gene, designated mob-5, an immediate transcriptional target of oncogenic ras. The invention further relates to the detection of mob-5 expression and/or the presence of the Mob-5 protein as potential markers for the early diagnosis of cancer.

2. Background Art

Oncogenic conversion of a normal cell into a tumor cell requires multiple genetic alterations (Land et al., 1983; Hunter, 1991). Of particular interest is the fact that mutations in Ras oncogenes cooperate with several other proto-oncogenes or mutant p53 tumor suppressor genes to transform mammalian cells (Hinds, et al. 1989). Ras proteins are among the most important molecular switch molecules that relay mitogenic or differentiation signals from the cell surface to the nucleus where selective genes are activated. Oncogenic mutations lock the Ras proteins into a permanent “on” position, leading to unregulated cell proliferation which is a hallmark of cancer. Mutations in the ras oncogene have been found at high frequency in a variety of human cancers, including those of gastrointestinal origin, such as pancreas and colon (Kiaris and Spandidos, 1995). It has been proposed that Ras proteins function, whether directly or through other signaling molecules, to control expression of genes that are important for cell growth and differentiation (Kern et al., 1991; Hunter, 1995). Progress has been made toward understanding Ras signaling pathway from growth factor receptor through activation of a cascade of protein kinases (Boguski and McCormick, 1993). In contrast, much less is known about the downstream genes that are transcriptionally activated by Ras.

This invention relates to the discovery of a novel gene, designated mob-5, as an immediate transcriptional target of oncogenic ras. The gene encodes a secreted protein of 183 amino acids, confirmed by imnmunoassays in oncogenic ras transformed cells and colorectal cancer cells from patient tissues. Normal ras activation cannot induce mob-5 expression.

Most of the current tumor markers are inadequate for the purpose of routine diagnosis because they are localized inside tumor cells or on the cell surface. The ideal diagnostic tumor marker should be detected using non-invasive procedures such as blood or urine tests instead of surgery. To this end, the best example has been the use of prostate specific antigen (PSA) as a diagnostic marker for prostate cancer. However, since PSA is expressed in both normal and cancerous prostate cells, the lack of tumor specificity has yielded a high rate of false positives which make the use of PSA as a diagnostic marker controversial. Unlike PSA, Mob-5 has tumor-specificity in addition to restricted expression in normal tissues. Therefore, the unique characteristics of tumor-specificity, specificity of activation by Ras, and secretion make mob-5 expression and/or Mob-5 presence markers for the early diagnosis of cancer.

### SUMMARY OF THE INVENTION

The invention provides an isolated nucleic acid comprising the nucleic acid set forth in the Sequence Listing as any of SEQ ID NO: 1, SEQ ID NO: 3 SEQ ID NO: 5, SEQ ID NO: 7 and SEQ ID NO: 9.

The invention also provides purified polypeptides having the sequence set forth in the Sequence Listing as any of SEQ ID NO: 2, SEQ ID NO: 4, SEQ ID NO: 6, SEQ ID NO: 8 and SEQ ID NO: 10.

This invention, in one aspect, relates to a method of detecting the presence of cancer in a patient comprising: a) contacting a sample from the patient with an antibody to Mob-5; and b) detecting the binding of the antibody with an antigen in the sample, wherein binding of antigen to the antibody indicates the presence of Mob-5 antigen in the sample and wherein Mob-5 antigen in the sample indicates the presence of cancer in the patient, thereby detecting the presence of cancer in the patient.

The invention further provides a method of classifying a cancer as a colorectal cancer comprising: a) obtaining a sample from a patient diagnosed with cancer; b) contacting the sample with an antibody to Mob-5; and c) detecting the binding of the antibody with an antigen in the sample, wherein the binding of the antibody to the antigen indicates the cancer is a colorectal cancer, thereby classifying the cancer as a colorectal cancer.

The invention also provides a method of classifying a cancer as a colorectal cancer comprising: a) obtaining a sample from a patient diagnosed with cancer; b) contacting the sample with a Mob-5 antigen; and c) detecting the binding of the antigen with an antibody in the sample, wherein the binding of the antigen to the antibody indicates the cancer is a colorectal cancer, thereby classifying the cancer as a colorectal cancer.

Further provided by this invention is a method of determining the effectiveness of an anti-cancer therapy comprising: a) obtaining a sample from a patient undergoing anti-cancer therapy, and b) monitoring the sample for expression of a mob-5 gene, whereby inhibition of the expression of the mob-5 gene indicates the anti-cancer therapy is effective, thereby determining the effectiveness of an anti-cancer therapy.

The invention also provides a method of determining the effectiveness of an anti-cancer therapy comprising: a) obtaining a sample from a patient undergoing anti-cancer therapy, and b) monitoring the sample for active mob-5 gene product, whereby inhibition of the activity of the mob-5 gene product indicates the anti-cancer therapy is effective, thereby determining the effectiveness of an anti-cancer therapy.

Also provided by this invention is a method of screening agents for anti-cancer activity comprising; a) administering the agent to a cancer cell; and b)monitoring the expression of a mob-5 gene in the cell, whereby an inhibition of the expression of the mob-5 gene indicates the agent has anti-cancer activity, thereby screening the agent for anti-cancer activity.

Further provided by this invention is a method of screening agents for anti-cancer activity comprising; a) administering the agent to a cancer cell; and b) monitoring the activity of a Mob-5 gene product in the cell, whereby an inhibition of the activity of the Mob-5 gene product indicates the agent has anti-cancer activity, thereby screening the agent for anti-cancer activity.

Also provided by this invention is a method of inhibiting a cellular transformation phenotype induced by an oncogene whose product functions upstream of a mob-5 gene product, comprising inhibiting formation of an active mob-5 gene product in a cell containing the upstream oncogene so as to inhibit the expression of the cellular transformation phenotype of the upstream oncogene.

Further provided is a method of inhibiting a cellular transformation phenotype induced by an oncogene whose product functions upstream of a mob-5 gene product, comprising inhibiting an active mob-5 gene product in a cell containing the upstream oncogene so as to inhibit the expression of the cellular transformation phenotype of the upstream oncogene.

Further provided by this invention is a method for inhibiting a mob-5 induced cellular transformation phenotype comprising inhibiting an active Mob-5 gene product in a cell so as to inhibit the expression of the mob-5 induced cellular transformation phenotype.

Further provided by this invention is a method of determining the effectiveness of an anti-cancer therapy comprising: a) obtaining a sample from a patient undergoing anti-cancer therapy, and b) monitoring the sample for the receptor for the mob-5 gene product, whereby a decreased amount of the receptor for the mob-5 gene product indicates the anti-cancer therapy is effective, thereby determining the effectiveness of an anti-cancer therapy.

Also provided by this invention is a method of determining the effectiveness of an anti-cancer therapy comprising: a) obtaining a sample from a patient undergoing anti-cancer therapy, and b) monitoring the sample for expression of a gene encoding a Mob-5 receptor, whereby inhibition of the expression of the gene encoding the Mob-5 receptor indicates the anti-cancer therapy is effective, thereby determining the effectiveness of an anti-cancer therapy.

The invention further provides a method of screening agents for anti-cancer activity comprising; a) administering the agent to a cancer cell; and b) monitoring the expression of a gene encoding a Mob-5 receptor in the cell, whereby an inhibition of the expression of the gene encoding a Mob-5 receptor indicates the agent has anti-cancer activity, thereby screening the agent for anti-cancer activity.

Also provided by this invention is a method of screening agents for anti-cancer activity comprising; a) administering the agent to a cancer cell; and b) monitoring the activity of a Mob-5 receptor in the cell, whereby an inhibition of the activity of the Mob-5 receptor indicates the agent has anti-cancer activity, thereby screening the agent for anti-cancer activity.

Further provided is a method of inhibiting a cellular transformation phenotype induced by an oncogene whose product functions upstream of a mob-5 gene product, comprising inhibiting formation of an active Mob-5 receptor in a cell containing the upstream oncogene so as to inhibit the expression of the cellular transformation phenotype of the upstream oncogene.

Further provided is a method of inhibiting a cellular transformation phenotype induced by an oncogene whose product functions upstream of a mob-5 gene product, comprising inhibiting an active Mob-5 receptor in a cell containing the upstream oncogene so as to inhibit the expression of the cellular transformation phenotype of the upstream oncogene.

Also provided is a method for inhibiting a mob-5 induced cellular transformation phenotype comprising inhibiting the formation of active Mob-5 receptor in a cell so as to inhibit the expression of the mob-5 induced cellular transformation phenotype.

The invention further provides a method for inhibiting a mob-5 induced cellular transformation phenotype comprising inhibiting an active Mob-5 receptor in a cell so as to inhibit the expression of the mob-5 induced cellular transformation phenotype.

The invention also provides a method of detecting the presence of cancer in a patient comprising: a) contacting a sample from the patient with an antibody to a Mob-5 receptor; and b) detecting the binding of the antibody with an antigen in the sample, wherein binding of antigen to the antibody indicates the presence of Mob-5 receptor antigen in the sample and wherein Mob-5 receptor antigen in the sample indicates the presence of cancer in the patient, thereby detecting the presence of cancer in the patient.

Further provided is a method of classifying a cancer as a colorectal cancer comprising: a) obtaining a sample from a patient diagnosed with cancer; b) contacting the sample with an antibody to a Mob-5 receptor; and c) detecting the binding of the antibody with an antigen in the sample, wherein the binding of the antibody to the antigen indicates the cancer is a colorectal cancer, thereby classifying the cancer as a colorectal cancer.

Also provided is a method of classifying a cancer as a colorectal cancer comprising: a) obtaining a sample from a patient diagnosed with cancer; b) contacting the sample with a Mob-5 receptor antigen; and c) detecting the binding of the antigen with an antibody in the sample, wherein the binding of the antigen to the antibody indicates the cancer is a colorectal cancer, thereby classifying the cancer as a colorectal cancer.

### BRIEF DESCRIPTION OF THE DRAWINGS

FIG. 1 illustrates the identification of mob-5 as an oncogenic ras target gene by differential display.

(A) Identification of mob-5 as a oncogenic h-ras inducible gene. Rat-1: iRas cells were treated with 2 mM IPTG to induce the oncogenic h-ras expression. Total cellular RNA isolated before and after IPTG induction at time points indicated were compared by differential display. The arrow indicates the mob-5 as a candidate gene induced by oncogenic ras.

(B) Identification of mob-5 as a gene activated by oncogenic h-ras but inactivated by MAP kinase inhibitor, PD98059. Total cellular RNA from the normal Rat-1 cells and their oncogenic h-ras transformed derivatives Rat-1(ras) treated with or without 10 μM of PD98059 for 24 hours were compared by differential display. The same primer combination as used in (A) identified the mob-5 (arrow) again as a candidate gene activated by ras but inhibited by ras downstream MAP kinase inhibitor. The tracing lines between (A) and (B) indicate the similar cDNA pattern displayed between the two independent screenings using the same primer combination.

(C) Northern blot confirmation of mob-5 as an oncogenic h-ras target gene. Total RNA from Rat-1: iRas cells before and after IPTG induction of h-ras was analyzed by Northern blot using mob-5 cDNA as a probe. Another ras target gene, mob-1, was also analyzed as a positive control. The induction of oncogenic h-Ras protein was analyzed by Western blot as indicated from the same cells. Note the induction of mutant h-Ras and mob-5 at 4 hours after IPTG addition.

(D) mob-5 expression cannot be activated by serum growth factors. Normal Rat-1 cells were starved for serum (0.5% BCS) for 48 hours and then stimulated with 10% BCS in the absence or presence of protein synthesis inhibitor, cyclohexanide as indicated. Total Cellular RNAs were analyzed by Northern blot using either mob-5 or mob-1 (as a positive control) cDNA probe.

FIG. 2 illustrates that the activation of mob-5 expression by oncogenic h-Ras is blocked by farnesyl transferase inhibitor (FTI)

(A) Rat-1(ras) cells were treated with 5 μM of FTI 739,749 for the period indicated and the total cellular RNA was analyzed by Northern blot using mob-5 cDNA as a probe. Ribosomal RNA is shown as a control for loading.

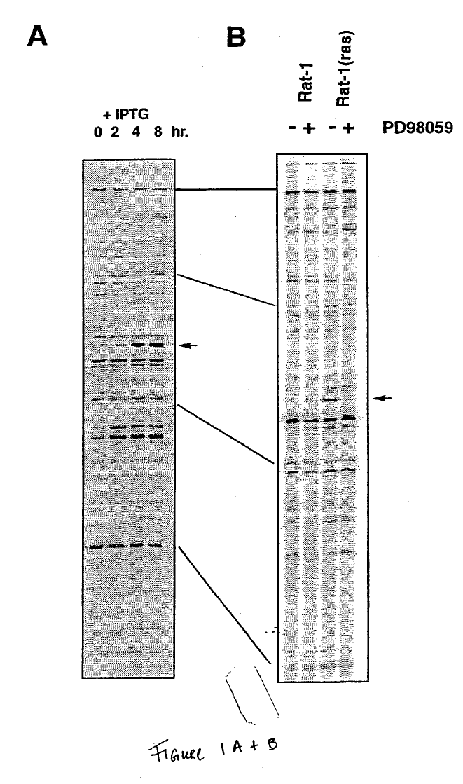

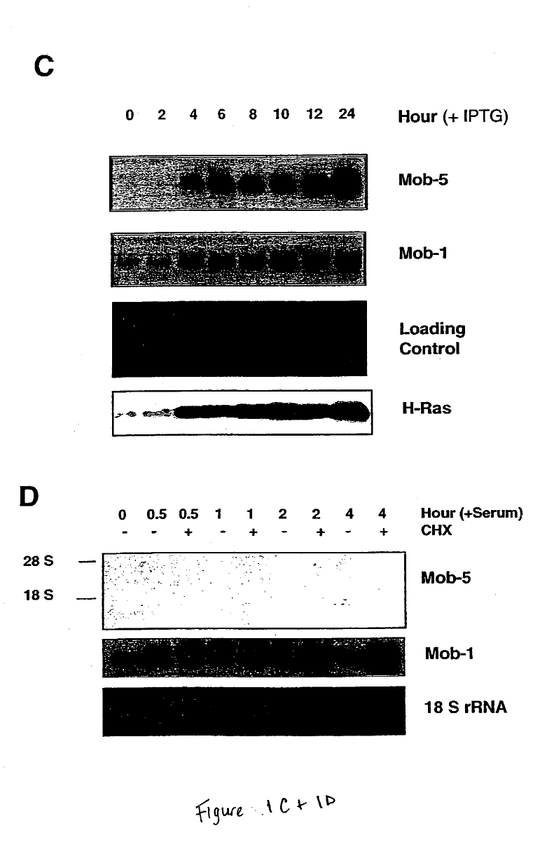

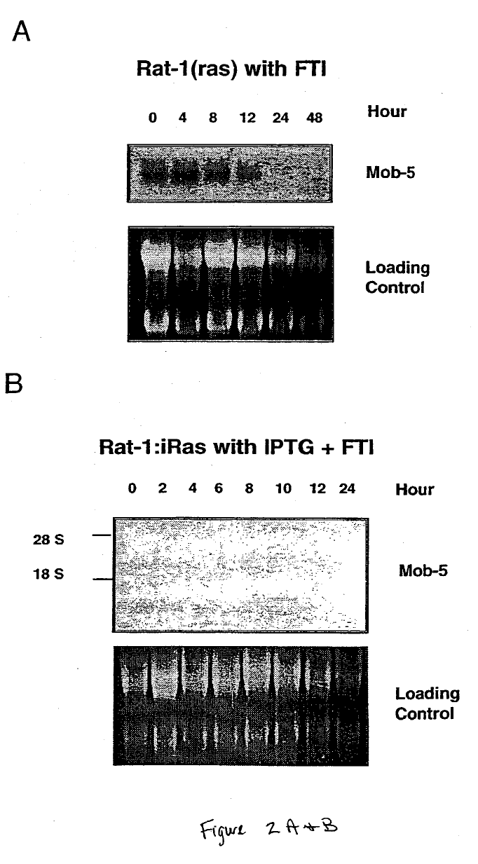

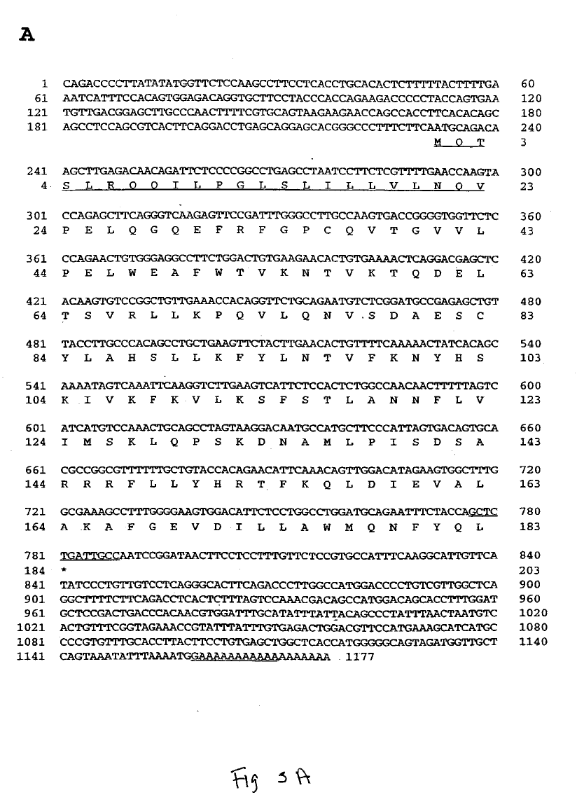

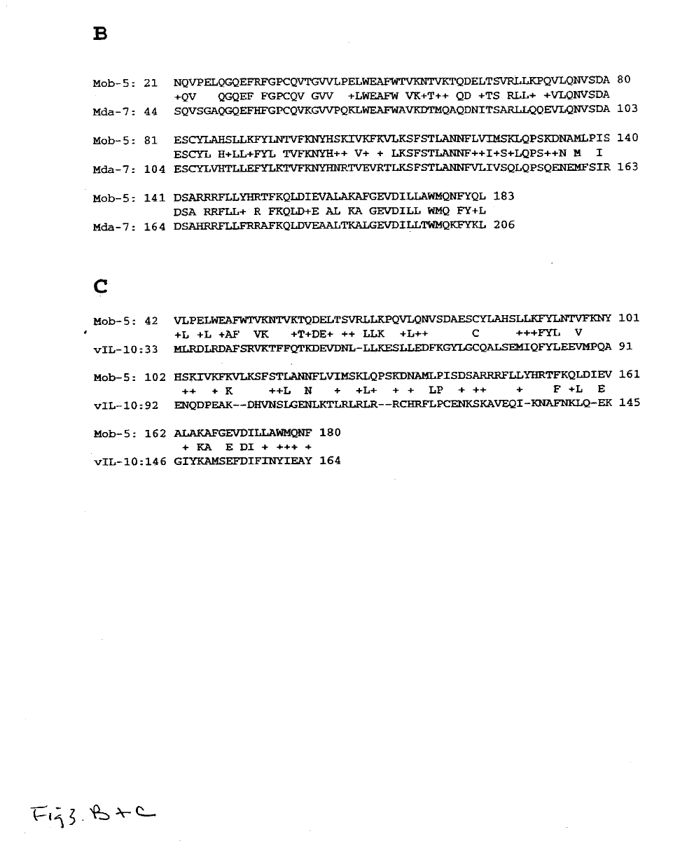

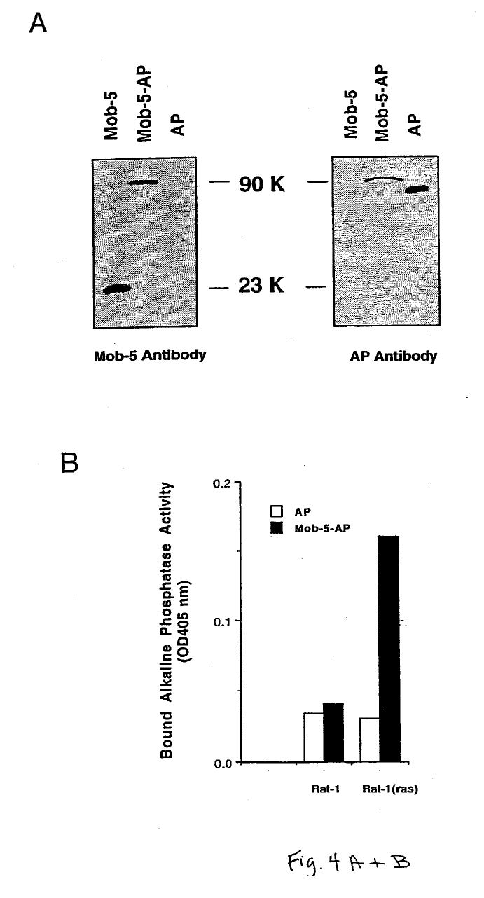

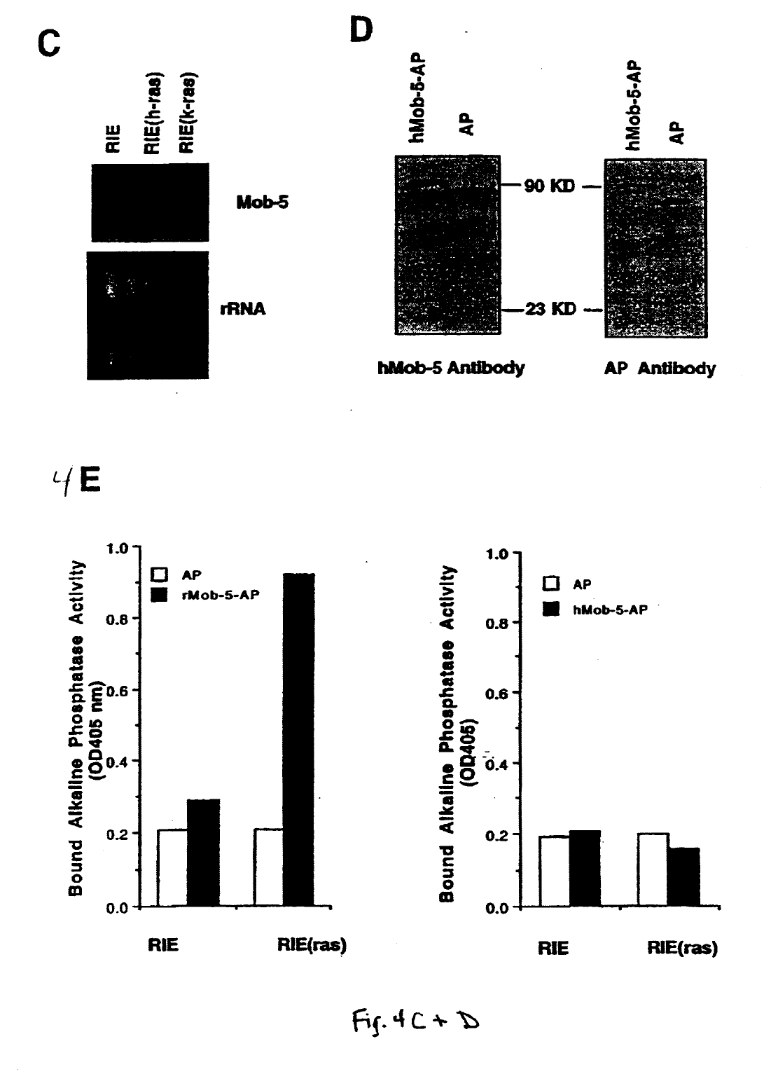

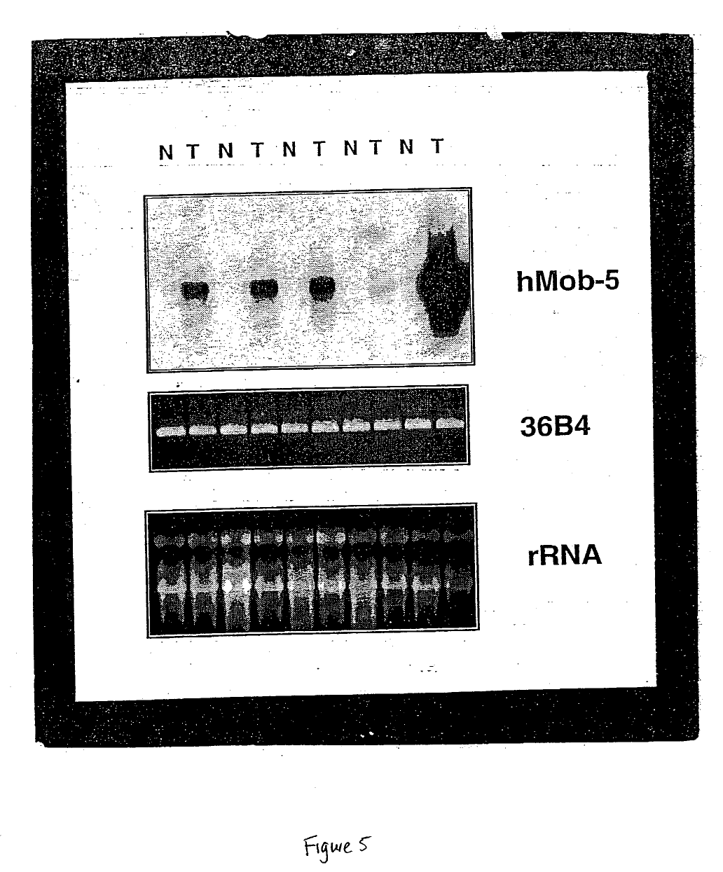

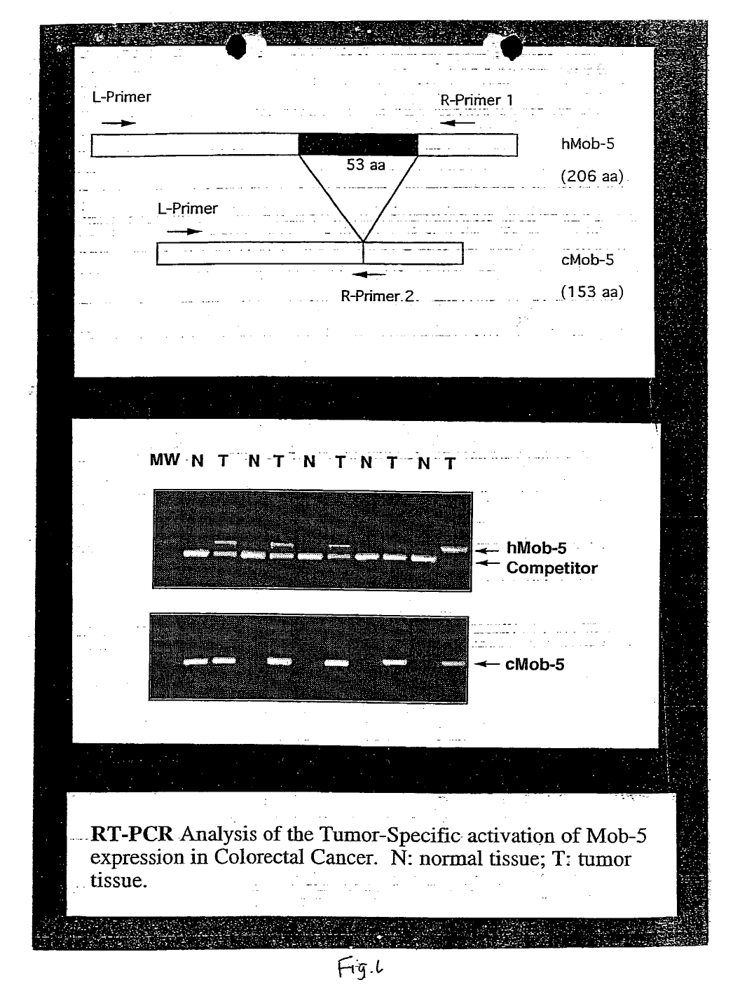

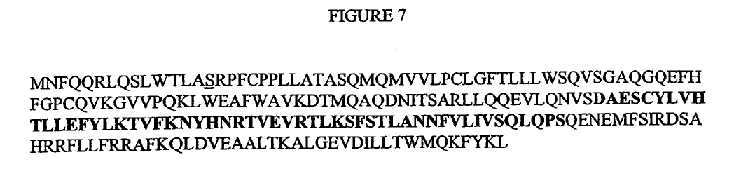

(B) The induction of mob-5 expression by oncogenic h-ras in Rat-1: iRas cells is blocked by FTI. The Rat-1: iRas cells were treated simultaneously with 2 mM IPTG and 5 μM FTI for the same period as FIG. 1C. Total RNAs were isolated and analyzed by Northern blot using mob-5 cDNA as a probe.

FIG. 3 illustrates the cDNA sequence of mob-5 and its predicted protein sequence alignment with Mda-7 and IL-10

(A) The full length cDNA of mob-5 and the predicted Mob-5 protein sequence. The full length cDNA of mob-5 was isolated from cDNA library of Rat-1(ras) cells using mob-5 cDNA probe from differential display (flanked by the arbitrary primer and anchored oligo-dT primer underlined at the 3′ untranslated region). The putative hydrophobic signal peptide located at the N-terminus of Mob-5 protein is also underlined.

(B) The protein sequence alignment of Mob-5 and Mda-7 was analyzed by the BLAST program from Genbank. Note, except for the N-terminal signal peptide sequence, the two proteins share 68 % identity over the rest of the entire coding sequences.

(C) The protein sequence alignment of Mob-5 and vIL-10 was analyzed by advanced BLAST program from Genbank. The entire coding regions share 23 % identity, except the non-conserved signal peptide sequences.

FIG. 4 illustrates Mob-5 expression can be activated by both oncogenic h-ras and k-ras and the gene encodes a 23 KD secreted protein which binds in a species-specific manner to the cell surface of oncogenic ras transformed fibroblasts and epithelial cells

(A) Expressing the native 23 KD Mob-5 in CRIP cells or the Mob-5-AP in 293T cells led to the secretion of Mob-5 and Mob-AP into the cell culture media. 30 μl of the culture media were analyzed by direct Western blot using polyclonal antibody to either Mob-5 or AP as indicated. The secreted 67 KD AP alone was used as control.

(B) Identification of a putative Mob-5 receptor also induced by h-ras oncogene. Both the normal and oncogenic h-ras transformed Rat-1 cells were analyzed for the ability to bind Mob-5-AP versus AP alone. The cell surface bound AP activity was measured to reveal a Mob-5 mediated-binding activity present only on the cell surface of the h-ras transformed cells. The result is representative of at least 5 independent experiments.

(C) Both oncogenic h-ras and k-ras can activate mob-5 expression in rat intestinal epithelial cells (RIE). Total cellular RNA from each cell line indicated were analyzed by Northern blot for the mob-5 expression using mob-5 cDNA as a probe.

(D) hMob-5 is also a secreted protein. The entire hMob-5 coding region with its native signal peptide sequence was fused at the C-terminus with AP and the hMob-5-AP from the cell culture medium was detected by direct Western blot analysis with antibody to either hMob-5 and AP. AP alone was used as a control for the antibody specificity.

(E) Mob-5-AP, but not hMob-5-AP, binds specifically to the cell surface of oncogenic h-ras transformed RIE cells. Both the normal and oncogenic h-ras transformed RIE cells were analyzed for the ability to bind Mob-5-AP versus AP alone. The same experiment was carried out with hMob-5-AP and AP alone. The cell surface bound AP activity was measured to reveal a species specific Mob-5 binding activity present only on the cell surface of the h-ras transformed RIE cells. The result is representative of at least 3 independent experiments.

FIG. 5 illustrates tumor specific expression of hMob-5. Total RNA from five colorectal cancer patients were analyzed by RT-PCR for the 800 bp coding region of hmob-5.

FIG. 6 illustrates the identification of cMob-5, a cancer-specific homolog of Mob-5 that has an internal deletion of 53 amino acids. CMob5 specific primers were used to detect cancer specific expression of cmob5 mRNA in colon cancer tissues.

FIG. 7 illustrates the protein sequence of human Mob-5 where the underlined S (Serine) is a single amino acid insertion between codon 14 and 15 of MDA-7, which makes human Mob-5 different from MDA-7. The bold amino acids correspond to the 53 amino acids that are deleted from the cancer-specific Mob-5 (cMob-5). cMob-5 does not contain the underlined serine amino acid residue.

### DETAILED DESCRIPTION OF THE INVENTION

The present invention may be understood more readily by reference to the following detailed description of the preferred embodiments of the invention and the Examples included therein.

Before the present compounds and methods are disclosed and described, it is to be understood that this invention is not limited to specific proteins, specific methods, or specific nucleic acids, as such may, of course, vary. It is also to be understood that the terminology used herein is for the purpose of describing particular embodiments only and is not intended to be limiting.

It must be noted that, as used in the specification and the appended claims, the singular forms “a,” “an,” and “the” include plural referents unless the context clearly dictates otherwise. Thus, for example, reference to “a nucleic acid” includes multiple copies of the nucleic acid and can also include more than one particular species of nucleic acid molecule.

The invention provides isolated nucleic acids comprising the nucleic acids set forth in the Sequence Listing as SEQ ID NO: 1 (corresponding to a cDNA encoding a rat Mob-5 protein), SEQ ID NO: 3 (corresponding to a cDNA encoding the cancer specific human Mob-5 protein homolog with an internal deletion of 53 amino acid residues, referred to as cMob-5.)and SEQ ID NO: 5 (corresponding to a cDNA encoding a human Mob-5 protein) SEQ ID NO: 7 (corresponding to a nucleic acid encoding a rat Mob-5-AP fusion protein and SEQ ID NO: 9 (corresponding to a nucleic acid encoding a human Mob-5-AP fusion protein.).

As used herein, the term “nucleic acid” refers to single-or multiple stranded molecules which may be DNA or RNA, or any combination thereof, including modifications to those nucleic acids. The nucleic acid may represent a coding strand or its complement, or any combination thereof. Nucleic acids may be identical in sequence to the sequences which are naturally occurring for any of the novel genes discussed herein or may include alternative codons which encode the same amino acid as that which is found in the naturally occurring sequence. These nucleic acids can also be modified from their typical structure. Such modifications include, but are not limited to, methylated nucleic acids, the substitution of a non-bridging oxygen on the phosphate residue with either a sulfur (yielding phosphorothioate deoxynucleotides), selenium (yielding phosphorselenoate deoxynucleotides), or methyl groups (yielding methylphosphonate deoxynucleotides).

The genes and nucleic acids provided for by the present invention may be obtained in any number of ways. For example, a DNA molecule encoding a Mob-5 protein can be isolated from the organism in which it is normally found. For example, a genomic DNA or cDNA library can be constructed and screened for the presence of the gene or nucleic acid of interest. Methods of constructing and screening such libraries are well known in the art and kits for performing the construction and screening steps are commercially available (for example, Stratagene Cloning Systems, La Jolla, Calif.). Once isolated, the gene or nucleic acid can be directly cloned into an appropriate vector, or if necessary, be modified to facilitate the subsequent cloning steps. Such modification steps are routine, an example of which is the addition of oligonucleotide linkers which contain restriction sites to the termini of the nucleic acid. General methods are set forth in Sambrook et al., “Molecular Cloning, a Laboratory Manual,” Cold Spring Harbor Laboratory Press (1989).

Once the gene or nucleic acid sequence of the desired Mob-5 protein is obtained, the sequence encoding specific amino acids can be modified or changed at any particular amino acid position by techniques well known in the art. For example, PCR primers can be designed which span the amino acid position or positions and which can substitute any amino acid for another amino acid. Then a nucleic acid can be amplified and inserted into the wild-type Mob-5 protein coding sequence in order to obtain any of a number of possible combinations of amino acids at any position of the Mob-5 protein. Alternatively, one skilled in the art can introduce specific mutations at any point in a particular nucleic acid sequence through techniques for point mutagenesis. General methods are set forth in Smith, M “In vitro mutagenesis” Ann. Rev. Gen., 19:423-462 (1985) and Zoller, M. J. “New molecular biology methods for protein engineering” Curr. Opin. Struct. Biol., 1:605-610 (1991). Techniques such as these can also be used to modify the genes or nucleic acids in regions other than the coding regions, such as the promoter regions for the Mob-5 protein. Likewise, these techniques can be used to alter the coding sequence without altering the amino acid sequence that is encoded. An example of a modified Mob-5 protein is cMob-5.

Another example of a method of obtaining a DNA molecule encoding a specific Mob-5 protein is to synthesize a recombinant DNA molecule which encodes the Mob-5 protein. For example, oligonucleotide synthesis procedures are routine in the art and oligonucleotides coding for a particular protein region are readily obtainable through automated DNA synthesis. A nucleic acid for one strand of a double-stranded molecule can be synthesized and hybridized to its complementary strand. One can design these oligonucleotides such that the resulting double-stranded molecule has either internal restriction sites or appropriate 5′ or 3′ overhangs at the termini for cloning into an appropriate vector. Double-stranded molecules coding for relatively large proteins can readily be synthesized by first constructing several different double-stranded molecules that code for particular regions of the protein, followed by ligating these DNA molecules together. For example, Cunningham, et al., “Receptor and Antibody Epitopes in Human Growth Hormone Identified by Homolog-Scanning Mutagenesis,” Science, 243:1330-1336 (1989), have constructed a synthetic gene encoding the human growth hormone gene by first constructing overlapping and complementary synthetic oligonucleotides and ligating these fragments together. See also, Ferretti, et al., Proc. Nat. Acad. Sci. 82:599-603 (1986), wherein synthesis of a 1057 base pair synthetic bovine rhodopsin gene from synthetic oligonucleotides is disclosed. By constructing a Mob-5 protein in this manner, one skilled in the art can readily obtain any particular Mob-5 protein with desired amino acids at any particular position or positions within the Mob-5 protein. See also, U.S. Pat. No. 5,503,995 which describes an enzyme template reaction method of making synthetic genes. Techniques such as this are routine in the art and are well documented. These nucleic acids or fragments of a nucleic acid encoding a Mob-5 protein can then be expressed in vivo or in vitro as discussed below.

The invention also provides for the isolated nucleic acids of SEQ ID NO: 1, SEQ ID NO: 3, SEQ ID NO: 5, SEQ ID NO: 7 and SEQ ID NO: 9 in a vector suitable for expressing the nucleic acid. Once a nucleic acid encoding a particular Mob-5 protein of interest, or a region of that nucleic acid, is constructed, modified, or isolated, that nucleic acid can then be cloned into an appropriate vector, which can direct the in vivo or in vitro synthesis of that wild-type and/or modified Mob-5 protein. The vector is contemplated to have the necessary functional elements that direct and regulate transcription of the inserted gene, or nucleic acid. These functional elements include, but are not limited to, a promoter, regions upstream or downstream of the promoter, such as enhancers that may regulate the transcriptional activity of the promoter, an origin of replication, appropriate restriction sites to facilitate cloning of inserts adjacent to the promoter, antibiotic resistance genes or other markers which can serve to select for cells containing the vector or the vector containing the insert, RNA splice junctions, a transcription termination region, or any other region which may serve to facilitate the expression of the inserted gene or hybrid gene. (See generally, Sambrook et al.).

There are numerous E. coli (Escherichia coli) expression vectors known to one of ordinary skill in the art which are useful for the expression of the nucleic acid insert. Other microbial hosts suitable for use include bacilli, such as Bacillus subtilis, and other enterobacteriaceae, such as Salmonella, Serratia, and various Pseudomonas species. In these prokaryotic hosts one can also make expression vectors, which will typically contain expression control sequences compatible with the host cell (e.g., an origin of replication). In addition, any number of a variety of well-known promoters will be present, such as the lactose promoter system, a tryptophan (Trp) promoter system, a beta-lactamase promoter system, or a promoter system from phage lambda. The promoters will typically control expression, optionally with an operator sequence, and have ribosome binding site sequences for example, for initiating and completing transcription and translation. If necessary, an amino terminal methionine can be provided by insertion of a Met codon 5′ and in-frame with the downstream nucleic acid insert. Also, the carboxy-terminal extension of the nucleic acid insert can be removed using standard oligonucleotide mutagenesis procedures.

Additionally, yeast expression can be used. There are several advantages to yeast expression systems. First, evidence exists that proteins produced in a yeast secretion systems exhibit correct disulfide pairing. Second, post-translational glycosylation is efficiently carried out by yeast secretory systems. The Saccharomyces cerevisiae pre-pro-alpha-factor leader region (encoded by the MF″-1 gene) is routinely used to direct protein secretion from yeast. (Brake, et al., “∝-Factor-Directed Synthesis and Secretion of Mature Foreign Proteins in Saccharomyces cerevisiae.” Proc. Nat. Acad. Sci., 81:4642-4646 (1984)). The leader region of pre-pro-alpha-factor contains a signal peptide and a pro-segment which includes a recognition sequence for a yeast protease encoded by the KEX2 gene: this enzyme cleaves the precursor protein on the carboxyl side of a Lys-Arg dipeptide cleavage signal sequence. The nucleic acid coding sequence can be fused in-frame to the pre-pro-alpha-factor leader region. This construct is then put under the control of a strong transcription promoter, such as the alcohol dehydrogenase I promoter or a glycolytic promoter. The nucleic acid coding sequence is followed by a translation termination codon which is followed by transcription termination signals. Alternatively, the nucleic acid coding sequences can be fused to a second protein coding sequence, such as Sj26 or beta-galactosidase, used to facilitate purification of the fusion protein by affinity chromatography. The insertion of protease cleavage sites to separate the components of the fusion protein is applicable to constructs used for expression in yeast. Efficient post translational glycosylation and expression of recombinant proteins can also be achieved in Baculovirus systems.

Mammalian cells permit the expression of proteins in an environment that favors important post-translational modifications such as folding and cysteine pairing, addition of complex carbohydrate structures, and secretion of active protein. Vectors useful for the expression of active proteins in mammalian cells are characterized by insertion of the protein coding sequence between a strong viral promoter and a polyadenylation signal. The vectors can contain genes conferring hygromycin resistance, gentamicin resistance, or other genes or phenotypes suitable for use as selectable markers, or methotrexate resistance for gene amplification. The chimeric protein coding sequence can be introduced into a Chinese hamster ovary (CHO) cell line using a methotrexate resistance-encoding vector, or other cell lines using suitable selection markers. Presence of the vector DNA in transformed cells can be confirmed by Southern blot analysis. Production of RNA corresponding to the insert coding sequence can be confirmed by Northern blot analysis. A number of other suitable host cell lines capable of secreting intact human proteins have been developed in the art, and include the CHO cell lines, HeLa cells, myeloma cell lines, Jurkat cells, etc. Expression vectors for these cells can include expression control sequences, such as an origin of replication, a promoter, an enhancer, and necessary information processing sites, such as ribosome binding sites, RNA splice sites, polyadenylation sites, and transcriptional terminator sequences. Preferred expression control sequences are promoters derived from immunoglobulin genes, SV40, Adenovirus, Bovine Papilloma Virus, etc. The vectors containing the nucleic acid segments of interest can be transferred into the host cell by well-known methods, which vary depending on the type of cellular host. For example, calcium chloride transformation is commonly utilized for prokaryotic cells, whereas calcium phosphate, DEAE dextran, or lipofectin mediated transfection or electroporation may be used for other cellular hosts.

Alternative vectors for the expression of genes or nucleic acids in mammalian cells, those similar to those developed for the expression of human gamma-interferon, tissue plasminogen activator, clotting Factor VIII, hepatitis B virus surface antigen, protease Nexin 1, and eosinophil major basic protein, can be employed. Further, the vector can include CMV promoter sequences and a polyadenylation signal available for expression of inserted nucleic acids in mammalian cells (such as COS-7).

Insect cells also permit the expression of mammalian proteins. Recombinant proteins produced in insect cells with baculovirus vectors undergo post-translational modifications similar to that of wild-type proteins. Briefly, baculovirus vectors useful for the expression of active proteins in insect cells are characterized by insertion of the protein coding sequence downstream of the Autographica californica nuclear polyhedrosis virus (AcNPV) promoter for the gene encoding polyhedrin, the major occlusion protein. Cultured insect cells such as Spodoptera frugiperda cell lines are transfected with a mixture of viral and plasmid DNAs and the viral progeny are plated. Deletion or insertional inactivation of the polyhedrin gene results in the production of occlusion negative viruses which form plaques that are distinctively different from those of wild-type occlusion positive viruses. These distinctive plaque morphologies allow visual screening for recombinant viruses in which the AcNPV gene has been replaced with a hybrid gene of choice. High quantity expression and production of the Mob-5 protein can also be achieved by transgenic animal technology by which animals can be made to produce Mob-5 in serum, milk, etc in large amounts.

The invention also provides for the vectors containing the contemplated nucleic acids in a host suitable for expressing the nucleic acids. The vectors containing the nucleic acid segments of interest can be transferred into host cells by well-known methods, which vary depending on the type of cellular host. For example, calcium chloride transformation, transduction, and electroporation are commonly utilized for prokaryotic cells, whereas calcium phosphate, DEAE dextran, or lipofection mediated transfection or electroporation may be used for other cellular hosts.

Alternatively, the genes or nucleic acids of the present invention can be operatively linked to one or more of the functional elements that direct and regulate transcription of the inserted gene as discussed above and the gene or nucleic acid can be expressed. For example, a gene or nucleic acid can be operatively linked to a bacterial or phage promoter and used to direct the transcription of the gene or nucleic acid in vitro. A further example includes using a gene or nucleic acid provided herein in a coupled transcription-translation system where the gene directs transcription and the RNA thereby produced is used as a template for translation to produce a polypeptide. One skilled in the art will appreciate that the products of these reactions can be used in many applications such as using labeled RNAs as probes and using polypeptides to generate antibodies or in a procedure where the polypeptides are being administered to a cell or a patient.

Expression of the gene or nucleic acid, in combination with a vector, can be by either in vivo or in vitro. In vivo synthesis comprises transforming prokaryotic or eukaryotic cells that can serve as host cells for the vector. Alternatively, expression of the gene or nucleic acid can occur in an in vitro expression system. For example, in vitro transcription systems are commercially available which are routinely used to synthesize relatively large amounts of mRNA. In such in vitro transcription systems, the nucleic acid encoding the Mob-5 protein would be cloned into an expression vector adjacent to a transcription promoter. For example, the Bluescript II cloning and expression vectors contain multiple cloning sites which are flanked by strong prokaryotic transcription promoters. (Stratagene Cloning Systems, La Jolla, Calif.). Kits are available which contain all the necessary reagents for in vitro synthesis of an RNA from a DNA template such as the Bluescript vectors. (Stratagene Cloning Systems, La Jolla, Calif.). RNA produced in vitro by a system such as this can then be translated in vitro to produce the desired Mob-5 protein. (Stratagene Cloning Systems, La Jolla, Calif.).

The present invention also contemplates nucleic acid probes for detecting the mob-5 gene wherein the DNA probe hybridizes to the nucleotide sequence set forth in the Sequence Listing as any of SEQ ID NO: 1, SEQ ID NO: 3, SEQ ID NO: 5, SEQ ID NO: 7 and/or SEQ ID NO: 9.

As used herein, the term “nucleic acid probe” refers to a nucleic acid fragment that selectively hybridizes under stringent conditions with a nucleic acid comprising a nucleic acid set forth in a sequence listed herein. This hybridization must be specific. The degree of complementarity between the hybridizing nucleic acid and the sequence to which it hybridizes should be at least enough to exclude hybridization with a nucleic acid encoding an unrelated protein.

Allelic variants can be identified and isolated by nucleic acid hybridization techniques. Probes selective to the nucleic acid set forth in the Sequence Listing as any of SEQ ID NO: 1, SEQ ID NO: 3, SEQ ID NO: 5, SEQ ID NO: 7 and SEQ ID NO: 9 can be synthesized and used to probe the nucleic acid from various cells, tissues, libraries etc. High sequence complementarity and stringent hybridization conditions can be selected such that the probe selectively hybridizes to allelic variants of the sequence set forth in the Sequence Listing as any of SEQ ID NO: 1, SEQ ID NO: 3,SEQ ID NO: 5, SEQ ID NO: 7 and SEQ ID NO: 9. For example, the selectively hybridizing nucleic acids of the invention can have at least 70%, 80%, 85%, 90%, 95%, 97%, 98% and 99% complementarity with the segment of the sequence to which it hybridizes. This percent complementarity can be based preferably on a nucleotide-by-nucleotide comparison of the two strands. The nucleic acids can be at least 12, 50, 100, 150, 200, 300, 500, 750, or 1000 nucleotides in length. Thus, the nucleic acid can be a coding sequence for the mob-5 proteins or fragments thereof that can be used as a probe or primer for detecting the presence of these genes. If used as primers, the invention provides compositions including at least two nucleic acids which hybridize with different regions so as to amplify a desired region. Depending on the length of the probe or primer, target region can range between 70% complementary bases and full complementarity and still hybridize under stringent conditions. For example, for the purpose of diagnosing the presence of an allelic variant of the sequence set forth in the Sequence Listing as any of SEQ ID NO: 1, SEQ ID NO: 3, and SEQ ID NO: 5 the degree of complementarity between the hybridizing nucleic acid (probe or primer) and the sequence to which it hybridizes is at least enough to distinguish hybridization with a nucleic acid from other bacteria. The invention provides examples of nucleic acids unique to SEQ ID NO: 1, SEQ ID NO: 3, SEQ ID NO: 5, SEQ ID NO: 7 and SEQ ID NO: 9 in the Sequence Listing so that the degree of complementarity required to distinguish selectively hybridizing from nonselectively hybridizing nucleic acids under stringent conditions can be clearly determined for each nucleic acid.

“Stringent conditions” refers to the washing conditions used in a hybridization protocol. In general, the washing conditions should be a combination of temperature and salt concentration chosen so that the denaturation temperature is approximately 5-20° C. below the calculated Tₘ of the nucleic acid hybrid under study. The temperature and salt conditions are readily determined empirically in preliminary experiments in which samples of reference DNA immobilized on filters are hybridized to the probe or protein coding nucleic acid of interest and then washed under conditions of different stringencies. The Tₘ of such an oligonucleotide can be estimated by allowing 2° C. for each A or T nucleotide, and 4° C. for each G or C. For example, an 18 nucleotide probe of 50% G+C would, therefore, have an approximate Tₘ of 54° C.

In another aspect, the invention provides the polypeptides encoded by the nucleic acids set forth in SEQ ID NO: 1, SEQ ID NO: 3, SEQ ID NO: 5 ,SEQ ID NO: 7 and SEQ ID NO: 9 as well as the polypeptides set forth in the Sequence Listing as SEQ ID NO: 2, SEQ ID NO: 4, SEQ ID NO: 6, SEQ ID NO: 8 and SEQ ID NO: 10. The invention also provides fragments of unmodified and modified Mob-5 proteins. Fragments of Mob-5 and cMob-5 that do not include a signal peptide sequence are also contemplated. The polypeptide fragments of the present invention can be recombinant proteins obtained by cloning nucleic acids encoding the polypeptide in an expression system capable of producing the polypeptide fragments thereof. For example, one skilled in the art can determine the active regions of a Mob-5 protein which can interact with another protein and cause a biological effect associated with the Mob-5 protein. In one example, amino acids found to not contribute to either the activity, binding specificity, or other biological effect associated with the Mob-5 protein can be deleted and/or substituted without a loss in the respective activity. The fragments, whether attached to other sequences or not, can also include insertions, deletions, substitutions, or other selected modifications of particular regions or specific amino acid residues, provided the activity of the peptide is not significantly altered or impaired. Further contemplated are polypeptides encoded by fragments of the Mob-5 nucleic acids provided herein.

These Mob-5 polypeptides can also be obtained in any of a number of procedures well known in the art. One method of producing a polypeptide is to link two peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to a particular protein can be synthesized by standard chemical reactions. For example, a peptide or polypeptide can be synthesized and not cleaved from its synthesis resin whereas the other fragment of a hybrid peptide can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form a larger polypeptide. (Grant, “Synthetic Peptides: A User Guide,” W. H. Freeman and Co., N.Y. (1992) and Bodansky and Trost, Ed., “Principles of Peptide Synthesis,” Springer-Verlag Inc., N.Y. (1993)). Alternatively, the peptide or polypeptide can be independently synthesized in vivo as described above. Once isolated, these independent peptides or polypeptides may be linked to form a larger protein via similar peptide condensation reactions.

For example, enzymatic ligation of cloned or synthetic peptide segments can allow relatively short peptide fragments to be joined to produce larger peptide fragments, polypeptides or whole protein domains (Abrahmsen et al. Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. “Synthesis of Proteins by Native Chemical Ligation,” Science, 266:776-779 (1994)). The first step is the chemoselective reaction of an unprotected synthetic peptide-α-thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site. Application of this native chemical ligation method to the total synthesis of a protein molecule is illustrated by the preparation of human interleukin 8 (IL-8) (Clark-Lewis et al. FEBS Lett., 307:97 (1987), Clark-Lewis et al., J.Biol.Chem., 269:16075 (1994), Clark-Lewis et al. Biochemistry,30:3128 (1991), and Rajarathnam et al. Biochemistry, 29:1689 (1994)).

Alternatively, unprotected peptide segments can be chemically linked where the bond formed between the peptide segments as a result of the chemical ligation is an unnatural (non-peptide) bond (Schnolzer et al. Science, 256:221 (1992)). This technique has been used to synthesize analogs of protein domains as well as large amounts of relatively pure proteins with full biological activity (deLisle Milton et al. “Techniques in Protein Chemistry IV,” Academic Press, New York, pp. 257-267 (1992)).

The Mob-5 polypeptides of this invention an also be fused to another protein such as alkaline phosphatase for detection methods. This fusion polypeptide can be utilized in an ELISA or Western blot to detect the receptor for Mob-5. The Mob5-alkaline phosphatase (Mob-5AP) polypeptides of this invention can be used to measure Mob-5 in an ELISA-based assay. For example, plates can be coated with an anti-Mob-5 antibody. Samples can then be added to the plates that contain a given amount of Mob-5AP which will bind to the plates. Therefore, in the absence of antigen, Mob-5AP should bind to the plates and produce the maximal amount of alkaline phosphatase activity. Upon addition of samples containing Mob-5, the antigen may compete with Mob-5AP for the Mob-5 antibody on the plates. The presence of antigen will competitively inhibit the binding of Mob-5AP.

Also provided herein are purified antibodies that selectively or specifically bind to the Mob-5 polypeptides provided and contemplated herein, for example, purified antibodies which selectively or specifically bind to a polypeptide encoded by the nucleic acid set forth in any of SEQ ID NO: 1, SEQ ID NO: 3, SEQ ID NO: 5 ,SEQ ID NO: 7 and SEQ ID NO: 9, as well as purified antibodies which selectively or specifically bind to the polypeptides set forth in SEQ ID NO: 2, SEQ ID NO: 4, SEQ ID NO: 6, SEQ ID NO: 8 and SEQ ID NO: 10. The antibody (either polyclonal or monoclonal) can be raised to any of the polypeptides provided and contemplated herein, both naturally occurring and recombinant polypeptides, and immunogenic fragments, thereof. The antibody can be used in techniques or procedures such as diagnostics, treatment, or vaccination. Anti-idiotypic antibodies and affinity matured antibodies are also considered.

The purified antibodies of this invention include monoclonal antibodies which can be used for diagnostic or analytical purposes. For example, the monoclonal antibody could be utilized in a clinical testing kit to monitor levels of Mob-5 in human tissues or secretions.

Antibodies can be made by many well-known methods (See, e.g. Harlow and Lane, “Antibodies; A Laboratory Manual” Cold Spring Harbor Laboratory, Cold Spring Harbor, N.Y., (1988)). Briefly, purified antigen can be injected into an animal in an amount and in intervals sufficient to elicit an immune response. Antibodies can either be purified directly, or spleen cells can be obtained from the animal. The cells can then fused with an immortal cell line and screened for antibody secretion. The antibodies can be used to screen nucleic acid clone libraries for cells secreting the antigen. Those positive clones can then be sequenced. (See, for example, Kelly et al. Bio/Technology, 10:163-167 (1992); Bebbington et al. Bio/Technology, 10:169-175 (1992)). Humanized and chimeric antibodies are also contemplated in this invention. Heterologous antibodies can be made by well known methods (See, for example, U.S. Pat. No. 5,545,806, 5,569,825, 5,625,126, 5,633,425, 5,661,016, 5,770,429, 5,789,650, and 5,814,318).

The phrase “specifically binds” with the polypeptide refers to a binding reaction which is determinative of the presence of the protein in a heterogeneous population of proteins and other biologics. Thus, under designated immunoassay conditions, the specified antibodies bound to a particular protein do not bind in a significant amount to other proteins present in the sample. Selective binding to an antibody under such conditions may require an antibody that is selected for its specificity for a particular protein. A variety of immunoassay formats may be used to select antibodies that selectively bind with a particular protein. For example, solid-phase ELISA immunoassays are routinely used to select antibodies selectively immunoreactive with a protein. See Harlow and Lane “Antibodies, A Laboratory Manual” Cold Spring Harbor Publications, New York, (1988), for a description of immunoassay formats and conditions that could be used to determine selective binding.

The present invention further provides a method of detecting the presence of cancer in a patient comprising: contacting a sample from the patient with an antibody to Mob-5, detecting the binding of the antibody with an antigen in the sample, wherein binding of the antigen to the antibody indicates the presence of Mob-5 antigen in the sample and wherein Mob-5 antigen in the sample indicates the presence of cancer in the patient, thereby detecting the presence of cancer in the patient.

A method of detecting the presence of cancer in a patient comprising: contacting a sample from the patient with Mob-5 antigen and detecting the binding of the antigen with an antibody in the sample, wherein binding of antibody to the antigen indicates the presence of Mob-5 antibody in the sample and wherein Mob-5 antibody in the sample indicates the presence of cancer in the patient, thereby detecting the presence of cancer in the patient.

The term “cancer,” when used herein refers to or describes the physiological condition, preferably in a mammalian subject, that is typically characterized by unregulated cell growth. Examples of cancer include but are not limited to ras-induced cancers, colorectal cancer, carcinoma, lymphoma, sarcoma, blastoma and leukemia. More particular examples of such cancers include squamous cell carcinoma, lung cancer, pancreatic cancer, cervical cancer, bladder cancer, hepatoma, breast cancer, prostrate carcinoma, rhabdomyosarcoma, colon carcinoma, and head and neck cancer. While the term “cancer” as used herein is not limited to any one specific form of the disease, it is believed that the methods of the invention will be particularly effective for cancers which are found to be accompanied by increased levels of Mob-5 expression.

The “subject” or “patient” of this method can be any animal. In a preferred embodiment, the animal of the present invention is a human. In addition, non-human animals which can be treated by the methods of this invention can include, but are not limited to, cats, dogs, birds, horses, cows, goats, sheep, guinea pigs, hamsters, gerbils and rabbits.

The sample of this invention can be from any organism and can be, but is not limited to, peripheral blood, plasma, urine, saliva, gastric secretion, feces, bone marrow specimens, primary tumors, embedded tissue sections, frozen tissue sections, cell preparations, cytological preparations, exfoliate samples (e.g., sputum), fine needle aspirations, amnion cells, fresh tissue, dry tissue, and cultured cells or tissue. It is further contemplated that the biological sample of this invention can also be whole cells or cell organelles (e.g., nuclei). The sample can be unfixed or fixed according to standard protocols widely available in the art and can also be embedded in a suitable medium for preparation of the sample. For example, the sample can be embedded in paraffin or other suitable medium (e.g., epoxy or acrylamide) to facilitate preparation of the biological specimen for the detection methods of this invention.

The term “antibody” is used herein in a broad sense and includes intact immunoglobulin molecules and fragments or polymers of those immunoglobulin molecules, so long as they exhibit any of the desired properties described herein. Antibodies are typically proteins which exhibit binding specificity to a specific antigen. Native antibodies are usually heterotetrameric glycoproteins, composed of two light (L) chains and two heavy (H) chains. The heavy and light chains are typically identical, but not necessarily so. Typically, each light chain is linked to a heavy chain by one or more covalent disulfide bond, while the number of disulfide linkages varies between the heavy chains of different immunoglobulin isotypes. Each heavy and light chain also typically has regularly spaced intrachain disulfide bridges. Each heavy chain typically has at one end a variable domain (V(H)) followed by a number of constant domains. Each light chain typically has a variable domain at one end (V(L)) and a constant domain at its other end; the constant domain of the light chain is typically aligned with the first constant domain of the heavy chain, and the light chain variable domain is typically aligned with the variable domain of the heavy chain. Particular amino acid residues are believed to form an interface between the light and heavy chain variable domains. The light chains of antibodies from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can typically be assigned to different classes. There are approximately five major classes of immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. The heavy chain constant domains that correspond to the different classes of immunoglobulins are called α, δ, ε, γ, and μ, respectively.

The term “variable” is used herein to describe certain portions of the variable domains which differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen binding site of antibodies (see Kabat E. A. et al., “Sequences of Proteins of Immunological Interest” National Institutes of Health, Bethesda, Md. (1987)). The constant domains are not involved directly in binding an antibody to an antigen, but exhibit various effector functions, such as participation of the antibody in antibody-dependent cellular toxicity.

The term “monoclonal antibody” as used herein refers to an antibody obtained from a substantially homogeneous population of antibodies, i.e., the individual antibodies comprising the population are identical except for possible naturally occurring mutations that may be present in minor amounts. The monoclonal antibodies herein specifically include “chimeric” antibodies in which a portion of the heavy and/or light chain may be identical with or homologous to corresponding sequences in antibodies derived from a particular species or belonging to a particular antibody class or subclass, while the remainder of the chain(s) may be identical with or homologous to corresponding sequences in antibodies derived from another species or belonging to another antibody class or subclass, as well as fragments of such antibodies, so long as they exhibit the desired antagonistic activity (See, U.S. Pat. No. 4,816,567 and Morrison et al., Proc. Natl. Acad. Sci. USA, 81:6851-6855 (1984)).

The present invention also provides a monoclonal antibody that binds to human Mob-5 and to rat Mob-5. This monoclonal antibody is produced from hybridoma clone L-14.

As used herein, “antigen” when used in the detection context generally means detecting the antigen, specifically Mob-5, cMob-5 (a cancer specific homolog of Mob-5 that has an internal deletion of 53 amino acids), the Mob-5 receptor, or a fragment thereof. The antigens of this invention can also be used to detect antibodies to Mob-5, the Mob-5 receptor or fragments thereof. In cancerous conditions, the antigen may exist on the cell surface as well as be detectable in a body fluid.

One example of the method of detecting the antigen is performed by contacting a fluid or tissue sample from the patient with an amount of a an antibody, possibly purified, reactive with the antigen, cells containing the antigen, or fragments of the antigen, and detecting the reaction of the antibody with the antigen. The fluid sample of this method can comprise any body fluid which would contain the antigen or a cell containing the antigen, such as blood, plasma, serum, saliva and urine, sputum, mucus and the like. An antibody used to detect the antigens of this invention is preferably specifically reactive with the antigen.

In the present invention, the step of detecting the binding of the antibody with the antigen can be further aided, in appropriate instances, by the use of a secondary antibody or other ligand which is reactive, either specifically with a different epitope or nonspecifically with the ligand or reacted antibody. The antibody can be labeled with a detectable marker.

Enzyme immunoassays such as immunofluorescence assays (IFA), enzyme linked immunosorbent assays (ELISA) and inmmunoblotting can be readily adapted to accomplish the detection of the antigen. An ELISA method effective for the detection of the antigen can, for example, be as follows: (1) bind the antibody to a substrate; (2) contact the bound antibody with a fluid or tissue sample containing the antigen; (3) contact the above with a secondary antibody bound to a detectable moiety (e.g., horseradish peroxidase enzyme or alkaline phosphatase enzyme); (4) contact the above with the substrate for the enzyme; (5) contact the above with a color reagent; (6) observe color change. Other assays for detecting the binding of an antibody to an antigen can be used.

The present invention further provides a kit for detecting the binding of an antibody to the Mob-5 or the Mob-5 receptor, or a fragment thereof. Particularly, the kit can detect the presence of an antigen specifically reactive with the antibody or an immunoreactive fragment thereof. The kit can include an antibody bound to a substrate, a secondary antibody reactive with the antigen and a reagent for detecting a reaction of the secondary antibody with the antigen. Such a kit can be an ELISA kit and can comprise the substrate, primary and secondary antibodies when appropriate, and any other necessary reagents such as detectable moieties, enzyme substrates and color reagents as described above. The diagnostic kit can, alternatively, be an immunoblot kit generally comprising the components and reagents described herein. The particular reagents and other components included in the diagnostic kits of the present invention can be selected from those available in the art in accord with the specific diagnostic method practiced in the kit. Such kits can be used to detect the binding of the antibody with Mob-5 and Mob-5 receptor, or a fragment thereof, in tissue and fluid samples from a patient.

One skilled in the art will be able to correlate the levels of Mob-5 antigen detected using the methods disclosed herein with a particular stage of the cancer, thus utilizing the detection method for prognostic purposes. The prognostic evaluation can determine what type of anti-cancer therapy to employ at different stages of cancer depending on the amounts of Mob-5 antigen detected in the patient's sample.

The invention also provides for a method of classifying a cancer as a colorectal cancer comprising, obtaining a sample from a patient diagnosed with cancer, contacting the sample with an antibody to Mob-5, and detecting the binding of the antibody with an antigen in the sample, wherein the binding of the antibody to the antigen indicated the cancer is colorectal cancer, thereby classifying the cancer as a colorectal cancer.

By “classifying” is meant to determine that the expression of Mob-5 corresponds to a specific type of cancer, such as colorectal cancer and not to another type of cancer, thus classifying the cancer. To ascertain that the cancer is, in fact, a colorectal cancer, the detection of Mob-5 in the sample can be combined with the detection of another specific tumor marker to confirm that the cancer is the specific type of cancer.

By “diagnosed” is meant that one skilled in the art has determined either by blood test, immunoassay, ultrasound, urinalysis, magnetic resonance imaging, physical examination, biopsy or any other diagnostic method that the patient has cancer.

The invention further provides a method of classifying a cancer as a colorectal cancer comprising, obtaining a sample from a patient diagnosed with cancer, contacting the sample with a Mob-5 antigen, and detecting the binding of the antigen with an antibody in the sample, wherein the binding of the antigen to the antibody indicates the cancer is a colorectal cancer, thereby classifying the cancer as a colorectal cancer. The detection of binding of Mob-5 antigen to Mob-5 antibody in the sample can be accomplished by utilizing immunological detection methods previously discussed or the capture immunoassay as described in the Examples.

Also provided by the present invention is a method of determining the effectiveness of an anti-cancer therapy comprising, obtaining a sample from a patient undergoing anti-cancer therapy, and monitoring the sample for expression of a mob-5 gene, whereby inhibition of the expression of the mob-5 gene indicates the anti-cancer therapy is effective, thereby determining the effectiveness of an anti-cancer therapy.

Also provided by the present invention is a method of determining the effectiveness of an anti-cancer therapy comprising, obtaining a sample from a patient undergoing anti-cancer therapy, and monitoring the sample for expression of a Mob-5 protein, such as Mob-5 or cMob-5 whereby inhibition of the expression of the Mob-5 or cMob-5 protein indicates the anti-cancer therapy is effective, thereby determining the effectiveness of an anti-cancer therapy.

Expression refers to the transcription and translation of a gene to yield the encoded protein, in particular a Mob-5 protein or a Mob-5 receptor protein . One of ordinary skill in the art will know how to monitor the level of expression of nucleic acids as well as proteins throughout the anti-cancer therapy in order to determine the effectiveness of an anti-cancer treatment. If the levels of the mob-5 gene or the activity of the Mob-5 protein decrease when compared to the levels measured prior to the anti-cancer therapy, the anti-cancer therapy may be effective. One skilled in the art may determine how effective the anti-cancer therapy is by correlating the decreases in mob-5 gene or Mob-5 protein expression with a particular stage of cancer. Similarly, decreases in mob-5 receptor gene or Mob-5 receptor expression can be correlated with a particular stage of cancer.

The nucleic acids of this invention can be detected with a probe capable of hybridizing to the nucleic acid of a cell or a sample. This probe can be a nucleic acid comprising the nucleotide sequence of a coding strand or its complementary strand or the nucleotide sequence of a sense strand or antisense strand, or a fragment thereof. The nucleic acid can comprise the nucleic acid of the mob-5 gene, or a sequence associated with a gene that is associated with the mob-5 gene, such as the mob-5 receptor gene. Thus, the probe of this invention can be either DNA or RNA and can bind either DNA or RNA, or both, in the biological sample. The probe can be the coding or complementary strand of a complete gene or gene fragment. The nucleotide sequence of the probe can be any sequence having sufficient complementarity to a nucleic acid sequence in the biological sample to allow for hybridization of the probe to the target nucleic acid in the biological sample under a desired hybridization condition. Ideally, the probe will hybridize only to the nucleic acid target of interest in the sample and will not bind non-specifically to other nucleic acids in the sample or other regions of the target nucleic acid in the sample. The hybridization conditions can be varied according to the degree of stringency desired in the hybridization. For example, if the hybridization conditions are for high stringency, the probe will bind only to the nucleic acid sequences in the sample with which it has a very high degree of complementarity. Low stringency hybridization conditions will allow for hybridization of the probe to nucleic acid sequences in the sample which have some complementarity but which are not as highly complementary to the probe sequence as would be required for hybridization to occur at high stringency. Since sequence divergence can exist between individuals for cancer or tumor-related genes, one skilled in the art can take these population differences into account when optimizing hybridization conditions. The hybridization conditions will therefore vary depending on the biological sample, probe type and target. An artisan will know how to optimize hybridization conditions for a particular application of the present method.

The nucleic acid probes of this invention can be modified nucleic acids. These modified nucleotides are well known in the art and include, but are not limited to, thio-modified deoxynucleotide triphosphates and borano-modified deoxynucleotide triphosphates (Eckstein and Gish, Trends in Biochent. Sci., 14:97-100 (1989) and Porter Nucleic Acids Research, 25:1611-1617 (1997)).

The nucleic acid probe can be commercially obtained or can be synthesized according to standard nucleotide synthesizing protocols well known in the art. Alternatively, the probe can be produced by isolation and purification of a nucleic acid sequence from biological materials according to methods standard in the art of molecular biology (Sambrook et al. 1989. Molecular Cloning: A Laboratory Manual, 2d Ed., Cold Spring Harbor Laboratory Pres, Cold Spring Harbor, N.Y.). The nucleic acid probe can be amplified according to well known procedure for amplification of nucleic acid (e.g., polymerase chain reaction). Furthermore, the probe of this invention can be linked to any of the detectable moieties of this invention by protocols standard in the art.

The detectable moieties to which the nucleic acid probe of this invention can be linked to include, but are not limited to, a hapten, biotin, digoxigenin, fluorescein isothiocyanate (FITC), dinitrophenyl, amino methyl coumarin acetic acid, acetylaminofluorene and mercury-sulfhydryl-ligand complexes, as well as any other molecule or compound which can be linked to a probe and detected either directly or indirectly according to the methods described herein. One skilled in the art will therefore appreciate that a probe, such as a nucleic acid probe or an antibody, can be labeled with a detectable moiety that can be directly detected, such as a flurorochrome or a dye, such as a chromogenic dye, and the use of secondary reagents to detect the probe is not strictly required.

It is further contemplated that the present invention also includes methods for oligonucleotide hybridization wherein the hybridized oligonucleotide is used as a primer for an enzyme catalyzed elongation reaction such as in situ PCR and primed in situ labeling reactions, whereby haptenized nucleotides are incorporated in situ. Additionally included are methods for in situ hybridization, employing synthetic peptide nucleic acid (PNA) oligonucleotide probes (Nielsen et al., 1991. “Sequence-selective recognition of DNA by strand displacement with a thymine-substituted polyamide.” Science 254:1497-1500; Egholm et al., 1993. “PNA hybridizes to complementary oligonucleotides obeying the Watson-Crick hydrogen bonding rules.” Nature 365:566-568).

The levels of protein in this invention can be detected by ELISA, FIA, immunoblotting or any other immunodetection method. These methods can be combined with histochemical or microscopic analysis to determine the levels of proteins in samples.

The patients of this invention undergoing anti-cancer therapy can include patients undergoing surgery, chemotherapy, radiotherapy, immunotherapy or any combination thereof. Examples of chemotherapeutic agents include cisplatin, 5-fluorouracil and S-1. Immunotherapeutics methods include administration of interleukin-2 and interferon-α.

Further provided by the present invention is a method of determining the effectiveness of an anti-cancer therapy comprising, obtaining a sample from a patient undergoing anti-cancer therapy, and monitoring the sample for active mob-5 gene product, whereby inhibition of the activity of the mob-5 gene product indicates the anti-cancer therapy is effective, thereby determining the effectiveness of an anti-cancer therapy.

By “active mob-5 gene product” is meant a product of the mob-5 gene which can exert a biological function associated with the mob-5 gene. An active mob-5 gene product may act as a transcriptional activator to elicit downstream effects. The mob-5 gene product may also function from outside of the cell either as a ligand which could bind to its cell surface receptor and participate in signal transduction, or it may function through the interaction with other secreted proteins and extracellular matrix molecules outside of cells. Any effect associated with mob-5 associated cancers is also contemplated by this invention.

The term “inhibition” is familiar to one skilled in the art and is used herein to describe any reduction in the activity of the mob-5 gene product. The degree of inhibition does not have to be complete, such as completely inhibiting the activity of the mob-5 gene product, and therefore comprises any inhibition of the activity of the mob-5 gene product relative to the activity of the mob-5 gene product in a similar environment in the absence of the inhibiting compound.

The invention also provides a method of screening agents for anti-cancer activity comprising, administering the agent to a cancer cell, monitoring the expression of a mob-5 gene in the cell, whereby an inhibition of the expression of the mob-5 gene indicates the agent has anti-cancer activity, thereby screening the agent for anti-cancer activity.

The “cells” of this invention include any cell type, cancerous or noncancerous, that may express or be affected by the expression of a mob-5 gene or the activity of a Mob-5 protein. Examples include, but are not limited to, colorectal cancer cells, pancreatic cancer cells, gastrointestinal cancer cells and prostate cancer cells.

Also provided is a method of screening agents for anti-cancer activity comprising, administering the agent to a cancer cell, monitoring the activity of a mob-5 gene product in the cell, whereby an inhibition of the activity of the mob-5 gene product indicates the agent has anti-cancer activity, thereby screening the agent for anti-cancer activity.

The invention also provides a method of inhibiting a cellular transformation phenotype induced by an oncogene whose product functions upstream of a mob-5 gene product, comprising inhibiting formation of an active mob-5 gene product in a cell containing the upstream oncogene so as to inhibit the expression of the cellular transformation phenotype of the upstream oncogene.

In this invention, “cellular transformation phenotype” means that cells have undergone at least some transformation characterized by one or more morphologic or biochemical changes such as loss of contact inhibition, increased rate of glycolysis, alterations of the cell surface and other changes that would be known to one skilled in the art.

The term “oncogene” refers to genes that produce products involved in altering cellular metabolism and often stimulate unregulated growth associated with malignant transformation of cells. Examples of oncogenes include but are not limited to, ras, src, myc, and fos. The cellular transformation phenotype of this invention can be induced by a ras oncogene product.

The term “inhibiting” in the context of gene expression, is familiar to one skilled in the art and is used herein to describe any compound or composition which inhibits or decreases the expression of an active mob-5 gene product. The degree of inhibition does not have to be complete, such as completely inhibiting the expression of a mob-5 gene product, and therefore comprises any inhibition of the expression of the mob-5 gene product relative to the expression of the mob-5 gene product in a similar environment in the absence of the inhibiting compound.

The cellular transformation phenotype of this invention can be inhibited by a nucleic acid antisense to the mob-5 gene. Antisense technology is well known in the art and describes a mechanism whereby a nucleic acid comprising a nucleotide sequence which is in a complementary, “antisense” orientation with respect to a coding or “sense” sequence of an endogenous gene, is introduced into a cell, whereby a duplex may form between the antisense sequence and its complementary sense sequence. The formation of this duplex may result in inactivation of the endogenous gene.

For example, the antisense nucleic acid can inhibit gene expression by forming an RNA/RNA duplex between the antisense RNA and the RNA transcribed from a target gene. The precise mechanism by which this duplex formation decreases the production of the protein encoded by the endogenous gene most likely involves binding of complementary regions of the normal sense mRNA and the antisense RNA strand with duplex formation in a manner that blocks RNA processing and translation. Alternative mechanisms include the formation of a triplex between the antisense RNA and duplex DNA or the formation of a DNA-RNA duplex with subsequent degradation of DNA-RNA hybrids by RNAse H. Furthermore, an antisense effect can result from certain DNA-based oligonucleotides via triple-helix formation between the oligomer and double-stranded DNA which results in the repression of gene transcription. Antisense nucleic acid can be produced for any relevant endogenous gene for which the coding sequence has been or can be determined according to well known methods.

A nucleic acid encoding an antisense RNA can be selected based on the protein desired to be inhibited or decreased in cells, by providing an RNA that will selectively bind to the cellular mRNA encoding such protein. Binding of the antisense molecule to the target mRNA may incapacitate the mRNAs, thus preventing its translation into a functional protein. The antisense RNA/mRNA complexes can then become a target for RNAse-H and are eventually degraded by the host cell RNAse-H. Control regions, such as enhancers and promoters, can be selected for antisense RNA targeting according to the cell or tissue in which it is to be expressed, as is known in the art. Preferable antisense-encoding constructs can encode full-length complements to target sequences; however, smaller length sequences down to oligonucleotide size can be utilized. For example, the antisense-encoding constructs can encode full-length complements to the mob-5 gene, smaller length sequences or oligonucleotide sequences.

The invention also provides a method of inhibiting a cellular transformation phenotype induced by an oncogene whose product functions upstream of a mob-5 gene product, comprising inhibiting an active mob-5 gene product in a cell containing the upstream oncogene so as to inhibit the expression of the cellular transformation phenotype of the upstream oncogene.

In the present invention, an active mob-5 gene product can be inhibited by an antibody to the mob-5 gene product. Also, the mob-5 gene product of this invention can be inhibited by ligand binding to the protein. Ligand binding includes reversible and nonreversible binding.

The present invention also provides a method for inhibiting a mob-5 induced cellular transformation phenotype comprising inhibiting the formation of active Mob-5 protein in a cell so as to inhibit the expression of the mob-5 induced cellular transformation phenotype. The formation of an active Mob-5 protein can be inhibited by a nucleic acid antisense to mob-5.

Further provided by this invention is a method for inhibiting a mob-5 induced cellular transformation phenotype comprising inhibiting an active Mob-5 protein in a cell so as to inhibit the expression of the mob-5 induced cellular transformation phenotype. An active Mob-5 protein in a cell can be inhibited by an antibody to Mob-5.

The present invention also provides a method of determining the effectiveness of an anti-cancer therapy comprising, obtaining a sample from a patient undergoing anti-cancer therapy, and monitoring the sample for the receptor for the mob-5 gene product, whereby a decreased amount of the receptor for the mob-5 gene product indicates the anti-cancer therapy is effective, thereby determining the effectiveness of an anti-cancer therapy.

The invention also provides a method of determining the effectiveness of an anti-cancer therapy comprising, obtaining a sample from a patient undergoing anti-cancer therapy, and monitoring the sample for expression of a gene encoding a Mob-5 receptor, whereby inhibition of the expression of the gene encoding the Mob-5 receptor indicates the anti-cancer therapy is effective, thereby determining the effectiveness of an anti-cancer therapy.

Further provided by the present invention is a method of screening agents for anti-cancer activity comprising, administering the agent to a cancer cell, monitoring the expression of a gene encoding a Mob-5 receptor in the cell, whereby an inhibition of the expression of the gene encoding a Mob-5 receptor indicates the agent has anti-cancer activity, thereby screening the agent for anti-cancer activity.

Further provided is a method of screening agents for anti-cancer activity comprising, administering the agent to a cancer cell, monitoring the activity of a Mob-5 receptor in the cell, whereby an inhibition of the expression of the Mob-5 receptor indicates the agent has anti-cancer activity, thereby screening the agent for anti-cancer activity.

Also provided is a method of inhibiting a cellular transformation phenotype induced by an oncogene whose product functions upstream of a mob-5 gene product, comprising inhibiting formation of an active Mob-5 receptor in a cell containing the upstream oncogene so as to inhibit the expression of the cellular transformation phenotype of the upstream oncogene. The cellular transformation phenotype can be induced by a ras oncogene product. The cellular transformation phenotype can be inhibited by a nucleic acid antisense to a Mob-5 receptor gene.

Also provided is a method of inhibiting a cellular transformation phenotype induced by an oncogene whose product functions upstream of a mob-5 gene product, comprising inhibiting an active Mob-5 receptor protein in a cell containing the upstream oncogene so as to inhibit the expression of the cellular transformation phenotype of the upstream oncogene. The cellular transformation phenotype can be induced by a ras oncogene product. An active Mob-5 receptor in a cell can be inhibited by an antibody to the Mob-5 receptor.

The present invention also provides a method for inhibiting a mob-5 induced cellular transformation phenotype comprising inhibiting the formation of active Mob-5 receptor in a cell so as to inhibit the expression of the mob-5 induced cellular transformation phenotype. A nucleic acid antisense to the Mob-5 receptor gene can be utilized to inhibit the formation of an active Mob-5 receptor.

Also provided by the present invention is a method for inhibiting a mob-5 induced cellular transformation phenotype comprising inhibiting an active Mob-5 receptor in a cell so as to inhibit the expression of the mob-5 induced cellular transformation phenotype. An active Mob-5 receptor can be inhibiting by an antibody to the Mob-5 receptor.

The present invention also provides a method of inhibiting a mob-5 induced cellular transformation phenotype, wherein the active Mob-5 receptor is inhibited by administering to the cell an altered Mob-5 which binds to the Mob-5 receptor, whereby binding of the altered Mob-5 to the receptor inhibits the binding of Mob-5 to the receptor, thereby inhibiting the expression of the mob-5 induced cellular transformation phenotype.

The invention also provides a method of detecting the presence of cancer in a patient comprising, contacting a sample from the patient with an antibody to a Mob-5 receptor, detecting the binding of the antibody with an antigen in serum, wherein binding of antigen to the antibody indicates the presence of Mob-5 receptor antigen in the sample and wherein Mob-5 receptor antigen in the sample indicates the presence of cancer in the patient, thereby detecting the presence of cancer in the patient.

The invention also provides a method of detecting the presence of cancer in a patient comprising, contacting a sample from the patient with a Mob-5 receptor antigen; detecting the binding of the antigen with an antibody in the sample, wherein binding of antigen to the antibody indicates the presence of Mob-5 receptor antibody in the sample and wherein Mob-5 receptor antibody in the sample indicates the presence of cancer in the patient, thereby detecting the presence of cancer in the patient.

The present invention also provides a method of detecting the Mob-5 receptor comprising, contacting a cell with a Mob5-AP fusion protein, detecting the binding of the Mob5-AP fusion protein to the cell, wherein binding of the Mob5-AP to the cell indicates the cell is a Mob-5 receptor expressing cell, thus detecting the Mob-5 receptor. Mob5AP fusion proteins can be made as described in the Examples and utilized in receptor binding assays described in the Examples and by Flanagan and Leder (1990) to determine if a cell expresses a cell surface receptor for a Mob-5 protein.

Further provided is a method of classifying a cancer as a colorectal cancer comprising, obtaining a sample from a patient diagnosed with cancer, contacting the sample with an antibody to a Mob-5 receptor, and detecting the binding of the antibody with an antigen in the sample, wherein the binding of the antibody to the antigen indicates the cancer is a colorectal cancer, thereby classifying the cancer as a colorectal cancer.

The present invention also provides a method of classifying a cancer as a colorectal cancer comprising, obtaining a sample from a patient diagnosed with cancer, contacting the sample with a Mob-5 receptor antigen, and detecting the binding of the antigen with an antibody in the sample, wherein the binding of the antigen to the antibody indicates the cancer is a colorectal cancer, thereby classifying the cancer as a colorectal cancer.

The present invention is more particularly described in the following examples which are intended as illustrative only since numerous modifications and variations therein will be apparent to those skilled in the art.

### EXAMPLES

Cell Culture All cell lines including Rat1, Rat1(ras), Rat-1: iRas (Liang et al., 1994; Zhang et al., 1996), RIE, RIE(h-ras) and RIE(k-ras) (Ko, 1995 ) were routinely grown in Dulbecco's Modified Eagle Medium (DMEM) (Life Technologies Inc. Grand Island, N.Y.) with 10% bovine calf serum (HyClone, Logan, Utah) and 1% penicillin-streptmoycin (Life Technologies Inc. Grand Island, N.Y.) at 37° C. with 10% CO₂. 293T cells and their derivatives were maintained under the same conditions as above except 10% fetal bovine serum (HyClone, Logan, Utah) was used in place of 10% bovine calf serum. Serum starvation and restimulation of the Rat-1 cells in the absence and presence of cyclohexamide (Sigma, St. Louis, Mo.) was carried out essentially as described previously (Zhang, et al., 1997).

RNA purification, automated differential display screening and Northern blot analysis Total RNA from cells were purified using the RNApure reagent following the manufacture instruction (GenHunter Corp., Nashville, Tenn.). DNase I treatment of RNA prior to differential display was carried out using the MessageClean kit (GenHunter Corp., Nashville, Tenn.). Differential display PCR reactions were setup in 96-well PCR plates (Perkin-Elmer) by the Beckman Biomek 2000 automated liquid dispensing workstation using one-base anchored oligo-dT primers and rationally designed arbitrary 13mers (Liang et al., 1994b) from the RNAimage kits (GenHunter Corp., Nashville, Tenn.). The cDNAs were amplified in the presence of ³³P [(α-dATP] (New England Nuclear, Boston, Mass.) using Taq DNA polymerase (Qiagene) and separated on 6 % denaturing polyacrylamide gels (National Diagnostic, Atlanta, Ga.).

Identification of mob-5 as an immediate target gene of oncogenic h-ras Using automated differential display screening with rational primer designs (Liang et al., 1994b), two paradigms, one with Rat-1: iRas cells containing IPTG inducible oncogenic h-ras (McCarthy et al., 1995; Zhang et al., 1997) , the other with oncogenic h-ras transformed Rat-1 cells before and after treatment of a MAP kinase inhibitor PD98059 (Pang et al., 1995), were set up for the systematic screening of oncogenic ras target genes. After screening through 90 combinations of primers representing 70% coverage of the genes expressed in a cell (Liang et al, 1995), a total of 14 ras inducible genes and 7 ras repressible genes were identified (Jo et. al, 1998). Four of the ras inducible genes, transin/stromelysin-1 (Matrisian, 1986), osteopontin (Craig et al., 1988), Cox-2 (Sheng et al., 1998) and Pai-2 (Cohen et al., 1989) were also identified by other methods in previous studies as ras targets. One of the 14 ras inducible genes, designated mob-5 was novel and the gene was identified in both screenings using either inducible ras oncogene or inhibitor which blocks ras signaling (FIGS. 1A and 1B). The 394 bp mob-5 cDNA was reamplified from the differential display gels, cloned and used as a probe to successfully verify the h-ras induction of the gene (FIG. 1C). Mob-1, a known ras target gene previously identified (Liang et al., 1994a; Zhang et al, 1997) was used as a control for ras induction. The IPTG treatment of Rat-1: iRas leads to the appearance of oncogenic H-Ras protein four hours post IPTG induction, with concomitant rapid induction of mob-5 mRNA. This result suggests that mob-5 is an early target gene of oncogenic h-ras.

Down-Regulation of Mob-5 Expression in Mutant H-Ras Transformed RIE-1 Cells by FTI

The tetrapeptide farnesyltransferase inhibitors from Merck have been shown to inhibit ras-mediated cell transformation of rat embryo fibroblasts. FTIs are able to inhibit the farnesylation of Ras protein, thereby preventing the membrane localization of Ras and its biological functions. If mob-5 is a relevant target gene in Ras transformation, FTIs should downregulate mob-5 expression in ras transformed RIE-1 cells. To test this assumption, exponentially growing RIE-1 cells transformed by mutant H-ras were treated with FTI 739,749 (10 μM); mob-5 mRNA expression and cellular morphology were monitored following drug addition. As predicted, mob-5 mRNA was downregulated as early as 24 hours after drug treatment and this downregulation was coupled temporally to reversion to more normal cellular morphology.

Mob-5 Cannot be Induced by Serum in Non-Transformed Rat-1 Cells, Nor Can Its Expression be Detected in Major Tissues from Normal Adult Rats

Previous studies showed that ras is required for the immediate early response to external growth factor stimulation and normal cell proliferation (Durkin and Whitefield, 1986; Taylor and Shalloway, 1996). Although mob-5 expression was not detected in nontransformed Rat-1 cells in continuous culture, one would predict that it could be expressed in response to serum stimulation if the gene is a downstream target of the Ras signaling pathway. Consistent with this prediction were findings that almost all of the previously identified ras target genes including mob-1 (Liang et al., 1994b), HB-EGF (McCarthy et al, 1995), Transin (Matrisian et al, 1985), osteopontin (Craig et al., 1988) and glucose transporter (Flier et al., 1987) could be induced by serum growth factors. To test the serum inducibility of the mob-5 expression, Rat-1 cells which do not express the mob-5 gene in continuous culture in the presence of serum were starved for serum and then restimulated with 10% fetal calf serum. Unlike mob-1, mob-5 expression was not detected following serum stimulation, either in the presence or absence of protein synthesis inhibitor (FIG. 1D). Northern blot analysis failed to detect any mob-5 expression in normal tissues from adult rats, including brain, heart, lung, kidney, stomach, pancreas, spleen, intestine, skin, skeletal muscle and blood.

Inhibition of Mob-5 Expression in Oncozenic H-Ras Transformed Rat-1 Cells by FTI.

The tetrapeptide farnesyltransferase inhibitors (FTI) have been shown to be able to inhibit h-ras-mediated cell transformation of rat embryo fibroblasts (Kohl et al., 1993). FTIs inhibit the farnesylation of Ras protein, thereby preventing its membrane localization and biological function. If mob-5 is indeed a target gene of ras, one would predict that FTIs would downregulate mob-5 expression in oncogenic h-ras transformed cells. To test this assumption, exponentially growing Rat-1(ras) cells were treated with 5 μM of FTI and the mob-5 mRNA expression and cellular morphology were monitored following the drug addition. As predicted, mob-5 mRNA expression was essentially abolished in 24 hours (FIG. 2A), when the cells begin to lose the transformed cellular morphology. Moreover, FTI was able to completely block the induction of mob-5 expression by IPTG in Rat-1: iRas cells (FIG. 2B).

mob-5 encodes a secreted protein Using the 394 bp cDNA probe obtained by differential display, the full length mob-5 cDNA was isolated from a cDNA library of h-ras transformed rat embryo fibroblasts (Liang et al., 1994a). The 1.2 kb mob-5 cDNA encodes an open reading frame of 183 amino acids with an in-frame stop codon 27 bases upstream of the putative translation initiation codon (FIG. 3A). As predicted, the 394 bp mob-5 cDNA sequence identified by differential display lies at the 3′ end of the mob-5 full length cDNA. The predicted Mob-5 protein exhibits 68 % sequence identity to Mda-7, a gene isolated as a differentiation associated gene from human melanoma cells treated with interferon-P and Mezerein (Jiang et al., 1995; FIG. 3B). The only other gene that showed significant degree of homology (23% identity) to Mob-5 over most of the coding regions is interleukin 10 (IL-10) which is an potent anti-inflammatory cytokine (FIG. 3C; Moore et al., 1990) The Mob-5 protein sequence contains a typical signal peptide sequence at its N-terminus, suggesting that Mob-5 is a secreted protein, although previous study failed to demonstrate that Mda-7 was secreted.

Over expression of the full length mob-5 cDNA with retroviral vector in a variety of rodent fibroblasts including Rat-1 cells led to high level secretion of Mob-5 protein with a predicted MW of 23 KD into the culture media, which could be readily detected by western blot using polyclonal antibody directed against the recombinant Mob-5 (FIG. 4A). However, over expression of Mob-5 alone in Rat-1 cells did not lead to any obvious morphological transformation elicited by oncogenic ras.

Identification of proteins interacting with Mob-5 by immunoprecipitation. As a secreted protein, Mob-5 may function from outside of the cell either as a ligand which could bind to its cell surface receptor and participate in signal transduction or it may function through the interaction with other secreted proteins and extracellular matrix molecules outside of cells. One approach is to determine if Mob-5 interacts with other proteins in vivo. K-ras and H-ras transformed RIE cells can be metabolically labeled with radioactive 35S-methionine and 35S-cysteine. The culture media of the cells will be immunoprecipitated with Mob-5 antibody. The precipitated protein(s) are then separated by SDS-PAGE and visualized by autoradiography to evaluate what other protein species are co-precipitated with Mob-5. As a negative control for the specificity of Mob-5 antibody, preimmune serum is used in parallel. To determine if the putative Mob-5 interacting protein(s) are present only in transformed cells, the same immunoprecipitation experiment will be carried out with normal RIE and Rat-1 cells. The protein(s) that specifically interact with Mob-5 may be further studied by affinity purification and microsequencing of the purified polypeptides. The obtained sequence information can then be either analyzed by database search for homology to known proteins or used to design degenerate oligonucleotide primers to amplify part of the corresponding cDNA of interest.

Identification of a putative receptor(s) of Mob-5 on the cell surface of h-ras transformed cells Another possible mechanism for Mob-5 function is that Mob-5 acts as an autocrine growth factor downstream of activated Ras. Ras transformed RIE cells can be used in a receptor binding assay. APtag technology (Flanagan and leder, 1990) was employed to create a Mob-5-alkaline phosphatase (AP) fusion protein, which can be used as an affinity agent for the receptor analysis. This strategy has been successfully used to identify and clone a number of important cell surface receptors, including those of leptin (Tartaglia et al., 1995) and semaphorin III (He and Tessier-Lavigne, 1997). Like Mob-5 itself, Mob-5-AP was secreted at high levels in 293T cells stably transfected with the recombinant plasmid. The secreted 90 KD Mob-5-AP can be readily detected from the culture media by either the alkaline phosphatase assay or direct Western blot analysis using anti-Mob-5 antibody (FIG. 4A). The construct of the Mob-5-AP fusion protein was also verified by Western blot using antibody to the KD human placental secreted AP (FIG. 4A, right panel). For the Mob-5 receptor study, Rat-1 cells before and after transformation by oncogenic h-ras were incubated with the cell culture media containing an equal amount of either the Mob-5-AP fusion protein or secreted AP itself. Unlike the AP alone control, Mob-5-AP was found to be preferentially bound to the cell surface of the h-ras transformed Rat-1 cells, but not of the parental control (FIG. 4B). Thus, Mob-5/Mob-5 receptor may represent a novel putative autocrine loop coordinately activated by ras oncogenes.

Activation of mob-5 expression by both oncogenic h-ras and k-ras in rat intestinal epithelial cells (RIE) To determine if mob-5 expression can be activated by other ras oncogenes or in different cell types, Northern blot analysis was conducted with RIE cells before and after transformation by oncogenic h-ras and k-ras (Ko et al., 1995). Both oncogenic h-ras and k-ras were shown to be able to activate mob-5 expression (FIG. 4C).

Over expression of hMob-5 in colon cancer and species specificity of the Mob-5R With a high degree of homology to Mob-5, it is likely that the hMob-5 is the human homolog of Mob-5. hMob-5 was found to be over expressed in nearly all human colon cancer specimens analyzed. Using RT-PCR, the entire coding region of hMob-5 was amplified from human colon cancer tissues and hMob-5-AP was constructed and expressed in 293T cells. Unlike previous studies on Mda-7, here the hMob-5-AP directed by hMob-5's native signal peptide was found to be secreted at a high level into the culture medium and could readily detected by both AP activity assay (range from 0.7 to 1.7 unit/ml) and direct Western blot using polyclonal antibody against hMob-5(FIG. 4D). Further confirmation of the 90 KD HMob-5-AP fusion protein was obtained by Western blot using antibody to AP itself (FIG. 4D).

As in Rat-1 cells, the putative Mob-5 receptor was also found to be differentially expressed between the normal and h-ras transformed RIE cells using Mob-5-AP fusion protein as a ligand (FIG. 4E, left panel). To determine whether hMob-5-AP could also differentially bind to the cell surface of h-ras transformed RIE cells, the receptor binding assay was conducted using hMob-5-AP in place of Mob-5-AP. Compared to the AP control, hMob-5-AP did not exhibit any preferential cell surface binding to the RIE cells before and after transformation by h-ras oncogene (FIG. 4E, right panel).

mob-5 expression is activated by both oncogenic h-ras and k-ras in intestinal epithelial cells Cancer arises from the epithelial cells rather than fibroblasts and K-ras mutations have been tightly linked with cancers of gastric intestinal origins (Kiaris and Spandidos, 1995). Supporting the importance of mob-5 in ras mediated oncogenesis, mob-5 expression was shown to be activated by both h-ras and k-ras oncogenes in rat intestinal epithelial cells (RIE). As in fibroblasts, Mob-5-AP binds specifically only to the ras transformed RIE cells but not to the non-transformed parental control. Cloning and sequencing of the full length mob-5 and hmob-5 cDNA Total RNA was isolated from h-Ras transformed rat embryo fibroblasts as described (22) and then further purified by polyA selection using polyATract mRNA isolation system (Promega, Madison, Wis.). The lambda ZAP II Vector/Gigapack cloning kit was used to construct the cDNA library following the instruction provided by the manufacturer. 500,000 plaques were screened for full length mob-5 cDNA using a ³²P-labeled cDNA probe from differential display. Positive plaques were excised as phagemids and sequenced by the molecular biology core facility of Dana-Farber Cancer Institute, Boston. The entire hmob-5 coding region was amplified from RNA isolated from human colon cancer tissues by RT-PCR based on the published sequence (Jiang et al., 1995). The cDNA synthesis was carried out in a total volume of 20μl containing up 1 μmg of DNA-free total RNA in the presence of 4μl of 5×RT buffer (GenHunter), with 20 μM dNTP, 1 μM of oligodT20 primer (GenHunter) and 200 units of MMLV reverse transcriptase (GenHunter) at 37° C. for 1 hour. After 5 min at 75 ° C. to inactivate the MMLV reverse transcriptase, 1/10 of the RT reaction equivalent to that was used as template for PCR amplification of hMob-5 using 0.2 μM of each LhMob-5 primer (5′-AAGATCTGAGGCTGCTT-3′) (SEQ ID NO: 11) and RhMob-5 primer(5′ CCAGATCTAGACATTCAGAGCTTGTAG-3′) (SEQ ID NO: 12). The PCR was carried out in 40μl containing 41 μl of 10×PCR buffer, 1.6 μl of 1.25 mM dNTP and 0.4 μl Taq polymerase (Qiagen). After an initial denaturation period of 3 min at 94° C., 30 cycles consisting of 15 seconds at 94° C., 40 seconds at 56° C. and 2 min at 72° C. followed. After the final 5 min extension at 72 ° C., the RT-PCR products were analyzed on a 1.5 % agarose gel. The amplified hMob-5 cDNA was cloned into the PCR-TRAP vector (GenHunter) and completely sequenced.

Cloning and Sequencing of the cancer-specific cMob-5 The cMob-5 cDNA was cloned by RT-PCR from colorectal cancer tissues. PCR primers used were as indicated in FIG. 6 (upper panel), L-Primer (5′-CCGCCTGTGTGCACTGTCTCTGATG-3′) (SEQ ID NO: 17)and R-Primer 1 (5′-CCGCCTGTGTGCACTGTCTCTGATG-3′) (SEQ ID NO: 18). In addition to the amplification of the expected hmob-5 cDNA of 484 bp, a smaller cDNA of 325 bp was discovered only from the colorectal cancer tissues but not from their adjacent normal controls. After cloning the smaller fragment into the PCR-TRAP cloning vector, DNA sequence analysis revealed an internal in-frame deletion of 159 base (53 amino acids) from the hMob-5 coding region. The full length cMob-5 cDNA was then obtained by RT-PCR from a colorectal cancer tissue using primer Lmda (5′-CCAGGAGGAACACGAGACTGAGAGATG-3′) (SEQ ID NO: 19) and primer Rmda (5′-GGAGGTCCAGGTCTAGACATTC-3′) (SEQ ID NO: 20).

For RT-PCR detection of cMob-5 specific mRNA message from cancer patient tissues (see FIG. 6), L-Primer (5′-TGCAAAGCCTGTGGACTTTAGCCAG-3′) (SEQ ID NO: 21) and cMob-5 specific primer R-Primer 2 (5′-GAAAACATCTCATTTTCTTCGAGAC-3′) (SEQ ID NO: 22)were used.

Expression and purification of the recombinant Mob-5 and HMob-5 proteins, and generation of polyclonal antibodies A 507-bp BamHI-HindIII fragment, containing the rat Mob-5 coding region without its N-terminal 23 signal peptides was generated by PCR using the cloned Mob-5 cDNA as a template. The primers used were Lhis (5′-CCAGAGCTTCAGGGTC-3′) (SEQ ID NO: 13) and Rhis (5′-AAGCTTTCCGGATTGGCAAT-3′) (SEQ ID NO: 14). The PCR product was first subcloned into PCR-TRAP vector (GenHunter, Nashville, Tenn.) and the BamHI-HindIII insert was then excised, purified and ligated into corresponding sites of the 6×-His tag expression vector pQE32 (Qiagen, Chatsworth, Calif.). For the expression recombinant hMob-5-6×His tagged protein, the coding region of hMob-, excluding the putative signal peptide sequence (codon 144), was amplified by PCR using primers Lhis-hMob5 (5′-GGATCCABBTATCAGGGG-3′)(SEQ ID NO: 15) and Rhis-hMob5 (5′-AAGCTTGTAGAATTTCTGCATC-3′) (SEQ ID NO: 16). The PCR product was cloned into the PCR-TRAP cloning vector and then excised as a BamH I-HindIII fragment before ligating into the corresponding sites of pQE32. The recombinant plasmids were transformed into Escherichia coli TG1 cells to produce N-terminal 6×-His tagged Mob-5 and hMob-5. Upon induction with 2 mM IPTG (Life Technology), the recombinant His-tagged Mob-5 and hMob-5 were overproduced as insoluble proteins in the bacteria and purified on a nickel-Sepharose column under denaturing conditions according to the manufacturer's protocol (Qiagen). The purified proteins were dialyzed in PBS to remove the denaturant and then injected into New Zealand White rabbits to produce polyclonal antibodies (HRP Inc., Denver, Pa.).

Construction of mob-5 mammalian expression construct A 680-bp BspHI-Bgl II restriction fragment containing the entire coding region of mob-5 was generated by PCR using cloned mob-5 cDNA as a template. The targeting construct was subsequently inserted between the Nco I and Bam I sites of retroviral vector pMFG-S (Danos and Mulligan, 1988; Dranoff, G. et al. 1993) and sequenced to ensure the sequence accuracy.

Mob-5-AP and hMob-5-AP fusion and receptor binding assay Both the entire Mob-5 and hMob-5 coding regions including their signal peptides were amplified by PCR and subsequently inserted into the Bgl II site of APtag2 cloning vector (Flanagan and Leder, 1990; GenHunter Corp., Nashville Tenn.) to allow the C-terminal fusion of the AP. The recombinant plasmids and APtag4 which express the secreted AP alone (Flanagan and Leder, 1990) were stably transfected into 293T cells. The expression of the fusion proteins, AP activity assay and receptor binding were essentially as described before (Flanagan and Leder, 1990 ). Specifically, the cells were seed at 5×10⁵ per well in 6-well plates in duplicates. After cells reach confluence, receptor binding assay was carried out with 1 ml of either the AP fusion protein- or AP alone-containing media ( all were 0.7 unit/ml) produced by the 293T cells.

Transfection and Retroviral Infection Recombinant plasmids were transfected into target cells by the standard calcium phosphate precipitation method. For retroviral packaging, pMFG-S-Mob-5 was cotransfected with pCMV-Neo vector at a rate of 10:1 (10 μg of pCMV-S-rMob-5: 1 μg of pCMV-Neo) into the CRIP packaging cells to produce G418 (1 mg/ml) resistant stable cell lines. Retroviral infection was carried out essentially as described previously (Zhang et al., 1998). For protein expression of Mob-5-AP, hMob-5-AP and AP alone, the corresponding plasmids were co-transfected with a puromycin-resistant vector, pBabe-puro, at a ratio of 10:1 and stable clones were selected by 10 μg/ml of puromycin.

Western Blot analysis Aliquots of 30μl of each cell culture medium were separated by electrophoresis on a sodium dodecyl sulfate-12% polyacrylamide gel (National Diagnostics, Atlanta, Ga.) and blotted onto a polyvinylidene difluoride membrane (Millipore, Bedford, Mass.). For immunoblotting, a 1:2000 dilution of either Mob-5-, hMob-5- or AP-specific antibodies (GenHunter Corp., Nashville, Tenn.) were used. The secondary antibody was anti-rabbit IgG conjugated with HRP (Amersham Life Science, Buckinghamshire, England). Western blot for Ras was carried out using anti-Ras antibody (Oncogene Science) and 50 μg/lane of total cellular protein extracted from Rat-1: iRas cells before and after IPTG treatment. Reactive proteins were visualized with ECL kit (Amersham Life Science, Buckinghamshire, England) following the manufacturer's protocol.

ELISA Assay for Mob-5

It was found that secreted Mob-5 (both human and rat proteins) binds to Gel Blue A (GBA) Sepharose beads at high affinity. This allows the use of GBA in a method for detecting Mob-5 in an ELISA. GBA is utilized to capture the antigen (i.e. Mob-5) and a biotinylated monoclonal antibody to Mob-5 is used to detect the antigen. The use of GBA allows for higher capacity and lower costs when compared to the traditional ELISA method. The method is performed by diluting a biotinylated Mab-hMob-5 (for example, the monoclonal antibody produced from hybridoma clone L-14) 1:500 in PBST (Phosphate Buffered Saline, Tween 0.05%); diluting Streptavidin-HRP (Sigma) 1:500 in PBST; mixing the diluted Mab-hMob-5 with the diluted Streptavidin-HRP in equal volume (for example, 500 ml each); letting the mixture sit for 5-10 minutes; washing the GBA beads with PBST twice; spinning and removing the upper liquid phase with an aspirator; resuspending the washed GBA beads in PBST as a 50% suspension; mixing 100-500 ml of a sample with 100 ml of the diluted Mab-hMob-5 and Streptavidin-HRP mixture in a tube; adding 50 ml of resuspended GBA beads to the tube; allowing the tube to rotate on a roller for 1-2 hours; spinning down the GBA beads for 2 minutes; aspirating the supernatant; washing the beads with 1 ml of PBST twice; washing the beads with dH₂0 twice; removing the supernatant; adding 150 ml of horseradish peroxidase (HRP) substrate with H₂O₂ to resuspend the beads; allowing the color to develop in the dark for as long as needed (from hours, to days or weeks). A hMob-5-9×His medium can be utilized as a positive control. One skilled in the art would know that variations of this protocol are possible. For example, the GBA beads may be added to the sample prior to the addition of the biotinylated Mab/streptavidin-HRP mixture, alkaline phosphatase labeled streptavidin can be used, wash times may vary, spin times may vary and development times may vary. By utilizing this method, representative results for a standard curve were obtained. These results were as follows:

| hMob-5 (ng/ml) | OD₄₀₅ |
|---|---|
| 0 | 0 |
| 2 | 0.216 |
| 10 | 0.941 |
| 20 | 1.881 |

Detection of auto-antibody to hMob-5 in colorectal cancer patients In order to substantiate the potential use of hMob-5 as a tumor diagnostic marker, the antibody capture immunoassay was used to detect the presence of antibody to Mob-5 in the plasma from colorectal cancer patients. In this assay, purified recombinant hMob-5 was blotted onto PVC membrane. After blocking the non-specific binding site with BSA, diluted plasma samples from normal controls and colorectal cancer patients obtained from Brigham-Women's Hospital and the Vanderbilt Clinic were incubated individually with the antigen. The presence of the auto-antibody to hMob-5 was detected after binding to the anti-human IgG labeled with HRP. About half of the colorectal cancer patients appeared to contain antibody to hMob-5, while 1 out of 8 normal controls showed a positive result.

Throughout this application, various publications are referenced. The disclosures of these publications in their entireties, as well as the references cited in these publications, are hereby incorporated by reference into this application in order to more fully describe the state of the art to which this invention pertains.

References

Barbacid, M. (1987). ras genes. Annu. Rev. Biochem. 56, 779-827.

Boguski, M. S. and McCormick, F. (1993). Proteins regulating Ras and its relatives. Nature 366, 643-654.

Cohen, L. R., J. Niclas, W. M. F. Lee, T-C. Wun, C. W. Crowley, A. D. Levinson, J. E. Sadler and Shuman, M. A. (1989). Effects of cellular transformation on expression of plasminogen activator inhibitor 1 and 2. J. Biol. Chem. 264, 8375-8383.

Craig A., M. Nemir, B. B. Mukherjee, A. F. Chambers and Denhardt, D. T. (1988). Identification of the major phosphoprotein secreted by many rodent cell lines as 2ar/osteopontin: enhance expression in H-ras-transformed 3T3 cells. Biochem. Biophys. Res. Commun. 157, 166-173.

Danos, O. and Mulligan, R. C. (1988). Safe and Efficient generation of recombinant retroviruses with amphotropic and ecotropic host ranges. Proc. Natl. Acad. Sci. U.S.A., 85, 6460-6464.

Dobrowolski, S., M. Harter and Stacey, D. W. (1994). Cellular ras activity is required for passage through multiple points of the G0/G1 Phase in BALB/c 3T3 cells. Mol. Cell. Biol. 14, 5441-5449.

Dranoff, G., E. Jaffee, A. Lazenby, P. Golumbek, H. Levitsky, K. Brose, V. Jackson, H. Hamada, D. Pardoll and Mulligan, R. C. (1993). Vaccination with irradiated tumor cells engineered to secrete Murine granulocyte-macrophage colony-stimulating factor stimulates potent, specific and long-lasting anti-tumor immunity. Proc. Natl. Acad. Sci. U.S.A., 90, 3539-3543.

Durkin, J. P., and Whitfield, J. F. (1986). Characterization of G1 transit induced by the mitogenic oncogenic Ki-ras gene product. Mol. Cell. Biol. 6, 1386-1392.

Flanagan, J. G. and Leder, P. (1990). The kit ligand: a cell surface molecule altered in steel mutant fibroblasts. Cell 63, 185-194.

Flier, J., M. M. Mueckler, P. Usher and Lodish, H. F. (1987). Elevated levels of glucose transport and transporter messenger RNA are induced by ras or src oncogenes. Science, 235, 1492-1495.

Godwin, A., and Lieberman, M. (1990). Early and late responses to induction of rasT24 expression in Rat1 cells. Oncogene 5, 1231-1241.

He, Z. and Tessier-Lavigne, M. (1997). Neuropilin is a receptor for the axonal chemorepellent semaphorin III. Cell 90, 739-751.

Jiang, H., Lin, J. J., Su, Z., Goldstein, N. I. and Fisher, P. B. (1995). Subtraction hybridization identifies a novel melanoma differentiation associated gene, mda-7, modulated during human melanoma differentiation, growth and progression. Oncogene 11, 2477-2486.

Jiang, H., Su, Z., Lin, J. J., Goldstein, N. I. Young, C. S. H. and Fisher, P. B. (1996). The melanoma differentiation associated gene mda-7 suppresses cancer cell growth. Proc. Natl. Acad. Sci. USA. 93, 9160-9165.

Jo, H., Zhang, H., Zhang, R. and Liang, P. (1998). Cloning oncogenic ras-regulated genes by differential display. Methods 16, 365-372.

Kiaris, H. and Spandidos, D. A. (1995). Mutation of ras genes in human tumors. Intl. J. Oncol. 7, 413-421.

Ko, T. C., Sheng, H. M., Reisman, D., Thompson, E. A. and Beauchamp, R. D. (1995). Transforming growth factor-beta 1 inhibits cyclin D1 expression in intestinal epithelial cells. Oncogene 10, 177-184.

Kohl, N. E., S. D. Mosser, S. J. deSolms, E. A. Giuliani, D. L. Pompiano, S. L. Graham, R. L. Smith, E. M. Scolnick, A. Oliff, and Gibbs, J. B. (1993). Selective inhibition of ras-dependent transformation by a farnesyltransferase inhibitor. Science 260:1934-1937.

Liang, P., and Pardee, A. B. (1992). Differential display of eukaryotic messenger RNA by means of the polymerase chain reaction. Science 257, 967-971.

Liang, P., L. Averboukh, W. Zhu, and Pardee, A. B. (1994a). Ras activation of novel genes: Mob-1 as a model. Proc. Natl. Acad. Sci. U.S.A., 91, 12515-12519.

Liang, P., Zhu, W., Zhang, X., Guo, Z., O'Connell, R., Averboukh, L., Wang, F. and Pardee, A. B. (1994b) Differential display using one-base anchored oligo-dT primer. Nucleic Acids Res. 22, 5763-5764.

Liang, P., D. Bauer, L. Averboukh, P. Warthoe, M. Rohrwild, H. Muller, M. Strauss and Pardee, A. B. (1995). Analysis of altered gene expression by differential display. Methods in Enzymol. 254, 304-321.

Liang, P. (1998). Factors ensuring successful use of differential display. Methods 16, 361-364.

Liu, Y., Wei, S. H. -Y., Ho, A. S. -Y., de Waal Malefyt, E. and Moore, K. W. (1994). Expression cloning and characterization of a human IL-10 receptor. J. Immunol. 152, 1821-1829.

Lowy, D. R. and Willumsen, B. M. (1993). Function and regulation of Ras. Annu. Rev. Biochem. 62, 851-891.

Matrisian, L. M., N. Glaichenhaus, Gesnel, M -C, and Breathnach, R. (1985). Epidermal growth factor and oncogenes induce transcription of the same cellular mRNA in rat fibroblasts. EMBO J., 4, 1435-1440.

McCarthy, S. A., M. L. Samuels, C. A. Pritchard, J. A. Abraham, and McMahon, M. (1995). Rapid induction of heparin-binding epidermal growth factor/diphtheria toxin receptor expression by Raf and Ras oncogenes. Genes & Dev. 9, 1953-1964.

Miyazaki, I., Cheung, R. K., Dosch, H. -M. (1993). Viral interleukin 10 is critical for the induction of B cell growth transformation by Epstein-Barr virus. J. Exp. Med. 178, 439447.

Moore, K. W., Vieira, P., Fiorentino, D. F., Trounstine, M. L., Khan, T. A. and Mosmann, T. R. (1990). Homology of cytokine synthesis inhibitory factor (IL-10) to Epstein-Barr virus gene BCRFI. Science 248, 1230-1234.

Moore, K. W., O'Garra, A., de Waal Malefyt, R., Vieira, P. and Mosmann, T. R. (1993). Interleukin-10. Annu. Rev. Immunol. 11, 165-190.

Pang, L., Sawada, T., Decker, S. J., Saltiel, A. R. (1995). Inhibition of MAP kinase kinase blocks the differentiation of PC-12 cells induced by nerve growth factors. J. Biol. Chem. 270, 13585-13588.

Sheng, H., Williams, C. S., Shao, J., Liang, P., Dubois, R. N, and Beauchamp, R. D. (1998). Induction of cyclooxygenase-2 by activated Ha-ras oncogene in Rat-1 fibroblasts and the role of mitogen-activated protein kinase pathway. J. Biol. Chem. 273, 22120-22127.

Soo, C., Shaw, W. W., Freymiller, E., Longaker, M. T., Bertolami, C. N., Chiu, R., Tieu, A. and Ting, K. (1999). Cutaneous rat wounds express C49a, a novel gene with homology to human melanoma differentiation associated gene, Mda-7. J. Cell. Biochem. 74, 1-10.

Tartaglia, L. A., Dembski, M., Weng, X., Deng, N., Culpepper, J., Dvos, R., Richards, G. J., Campfield, L. A., Clark, F. T., Deeds, J., Muir, C., Sanker, S., Moriarty, A., Moore, K. J., Smutko, J. S., Mays, G. G., Woolf, E. A., Monroe, C. A. and Tepper, R. I. (1995). Identification and expression cloning of a leptin receptor, OB-R. Cell 83, 1263-1271.

Taylor, S. J. and Shalloway, D. (1996). Cell cycle-dependent activation of Ras. Curr. Biol. 6, 1621-1627.

Zhang, R., Zhang, H., Zhu, W., Coffey, R., Pardee, A. B. and Liang, P. (1997). Mob-1, a ras target gene, is over expressed in colorectal cancer. Oncogene 14, 1607-1610.

Zhang, R., Averboukh, L., Zhu, W., Zhang, H., Jo, H., Dempsey, P. J., Coffey, R., A. Pardee, A. B. and Liang, P. (1998). Identification of rCop-1, a New Member of CCN Gene Family, as a Negative Regulator for Cell Transformation. Mol. Cell. Biol., 18, 6131-6141.

What is claimed is:

1. An isolated nucleic acid comprising the nucleic acid set forth in the Sequence Listing as SEQ ID NO: 1.

2. The isolated nucleic acid of claim 1 in a vector suitable for expressing the nucleic acid.

3. The vector of claim 2 in a host suitable for expressing the nucleic acid.

4. A purified polypeptide encoded by the nucleic acid of claim 1.

5. An isolated nucleic acid encoding the polypeptide of claim 4.

6. The polypeptide of claim 4, having the sequence set forth in the Sequence Listing as SEQ ID NO: 2.

7. A purified antibody which specifically binds to the polypeptide of claim 6.

8. An isolated nucleic acid comprising the nucleic acid set forth in the Sequence Listing as SEQ ID NO: 3.

9. The isolated nucleic acid of claim 8 in a vector suitable for expressing the nucleic acid .

10. The vector of claim 9 in a host suitable for expressing the nucleic acid.

11. A purified polypeptide encoded by the nucleic acid of claim 8.

12. An isolated nucleic acid encoding the polypeptide of claim 11.

13. The polypeptide of claim 12, having the sequence set forth in the Sequence Listing as SEQ ID NO: 4.

14. A purified antibody which specifically binds to the polypeptide of claim 13.

15. An isolated nucleic acid comprising the nucleic acid set forth in the Sequence Listing as SEQ ID NO: 5.

16. The isolated nucleic acid of claim 15 in a vector suitable for expressing the nucleic acid.

17. The vector of claim 16 in a host suitable for expressing the nucleic acid.

18. A purified polypeptide encoded by the nucleic acid of claim 15.

19. An isolated nucleic acid encoding the polypeptide of claim 18.

20. The polypeptide of claim 18, having the sequence set forth in the Sequence Listing as SEQ ID NO: 6.

21. A purified antibody which specifically binds to the polypeptide of claim 20.

22. The isolated nucleic acid of claim 1 comprising the nucleic acid set forth in the Sequence Listing as SEQ ID NO: 7.

23. The isolated nucleic acid of claim 22 in a vector suitable for expressing the nucleic acid.

24. The vector of claim 23 in a host suitable for expressing the nucleic acid.

25. A purified polypeptide encoded by the nucleic acid of claim 22.

26. An isolated nucleic acid encoding the polypeptide of claim 25.

27. The polypeptide of claim 26, having the sequence set forth in the Sequence Listing as SEQ ID NO: 8.

28. A purified antibody which specifically binds to the polypeptide of claim 27.

29. The isolated nucleic acid of claim 15 comprising the nucleic acid set forth in the Sequence Listing as SEQ ID NO: 9.

30. The isolated nucleic acid of claim 29 in a vector suitable for expressing the nucleic acid.

31. The vector of claim 30 in a host suitable for expressing the nucleic acid.

32. A purified polypeptide encoded by the nucleic acid of claim 29.

33. An isolated nucleic acid encoding the polypeptide of claim 32.

34. The polypeptide of claim 32, having the sequence set forth in the Sequence Listing as SEQ ID NO: 10.

35. A purified antibody which specifically binds to the polypeptide of claim 34.

36. A method of detecting the presence of cancer in a patient comprising:
a. contacting a sample from the patient with an antibody to Mob-5; and
b. detecting the binding of the antibody with an antigen in the sample, wherein binding of antigen to the antibody indicates the presence of Mob-5 antigen in the sample and wherein Mob-5 antigen in the sample indicates the presence of cancer in the patient, thereby detecting the presence of cancer in the patient.

37. The method of claim 36 wherein the cancer is a ras-induced cancer.

38. The method of claim 36, wherein the cancer is a colorectal cancer.

39. A method of detecting the presence of cancer in a patient comprising:
a. contacting a sample from the patient with Mob-5 antigen; and
b. detecting the binding of the antigen with an antibody in the sample, wherein binding of antibody to the antigen indicates the presence of Mob-5 antibody in the sample and wherein Mob-5 antibody in the sample indicates the presence of cancer in the patient, thereby detecting the presence of cancer in the patient.

40. The method of claim 39 wherein the cancer is a ras-induced cancer.

41. The method of claim 39, wherein the cancer is a colorectal cancer.

42. A method of determining the effectiveness of an anti-cancer therapy comprising:
a. obtaining a sample from a patient undergoing anti-cancer therapy, and b. monitoring the sample for expression of a mob-5 gene, whereby inhibition of the expression of the mob-5 gene indicates the anti-cancer therapy is effective, thereby determining the effectiveness of an anti-cancer therapy.

43. A method of determining the effectiveness of an anti-cancer therapy comprising:
a. obtaining a sample from a patient undergoing anti-cancer therapy, and
b. monitoring the sample for active mob-5 gene product, whereby inhibition of the activity of the mob-5 gene product indicates the anti-cancer therapy is effective, thereby determining the effectiveness of an anti-cancer therapy.

44. A method of screening agents for anti-cancer activity comprising;
a. administering the agent to a cancer cell;
b. monitoring the expression of a mob-5 gene in the cell, whereby an inhibition of the expression of the mob-5 gene indicates the agent has anti-cancer activity, thereby screening the agent for anti-cancer activity.

45. A method of screening agents for anti-cancer activity comprising;
a. administering the agent to a cancer cell;
b. monitoring the activity of a Mob-5 gene product in the cell, whereby an inhibition of the activity of the Mob-5 gene product indicates the agent has anti-cancer activity, thereby screening the agent for anti-cancer activity.

46. A method of inhibiting a cellular transformation phenotype induced by an oncogene whose product functions upstream of a mob-5 gene product, comprising inhibiting formation of an active mob-5 gene product in a cell containing the upstream oncogene so as to inhibit the expression of the cellular transformation phenotype of the upstream oncogene.

47. The method of claim 46, wherein the cellular transformation phenotype is inhibited by a nucleic acid antisense to the mob-5 gene.

48. The method of claim 46, wherein the cellular transformation phenotype is induced by a ras oncogene product.

49. A method of inhibiting a cellular transformation phenotype induced by an oncogene whose product functions upstream of a mob-5 gene product, comprising inhibiting an active mob-5 gene product in a cell containing the upstream oncogene so as to inhibit the expression of the cellular transformation phenotype of the upstream oncogene.

50. The method of claim 49, wherein the mob-5 gene product is inhibited by an antibody to the mob-5 gene product.

51. The method of claim 49, wherein the cellular transformation phenotype is induced by a ras oncogene product.

52. A method for inhibiting a mob-5 induced cellular transformation phenotype comprising inhibiting the formation of active Mob-5 gene product in a cell so as to inhibit the expression of the mob-5 induced cellular transformation phenotype.

53. The method of claim 52, wherein the inhibition of the formation of an active Mob-5 protein is by a nucleic acid antisense to mob-5.

54. A method for inhibiting a mob-5 induced cellular transformation phenotype comprising inhibiting an active Mob-5 gene product in a cell so as to inhibit the expression of the mob-5 induced cellular transformation phenotype.

55. The method of claim 54, wherein the active Mob-5 gene product is inhibited by an antibody to Mob-5.

56. A method of determining the effectiveness of an anti-cancer therapy comprising:
a. obtaining a sample from a patient undergoing anti-cancer therapy, and
b. monitoring the sample for the receptor for the mob-5 gene product, whereby a decreased amount of the receptor for the mob-5 gene product indicates the anti-cancer therapy is effective, thereby determining the effectiveness of an anti-cancer therapy.

57. A method of determining the effectiveness of an anti-cancer therapy comprising:
a. obtaining a sample from a patient undergoing anti-cancer therapy, and
b. monitoring the sample for expression of a gene encoding a Mob-5 receptor, whereby inhibition of the expression of the gene encoding the Mob-5 receptor indicates the anti-cancer therapy is effective, thereby determining the effectiveness of an anti-cancer therapy.

58. A method of screening agents for anti-cancer activity comprising;
a. administering the agent to a cancer cell;
b. monitoring the expression of a gene encoding a Mob-5 receptor in the cell, whereby an inhibition of the expression of the gene encoding a Mob-5 receptor indicates the agent has anti-cancer activity, thereby screening the agent for anti-cancer activity.

59. A method of screening agents for anti-cancer activity comprising;
a. administering the agent to a cancer cell;
b. monitoring the activity of a Mob-5 receptor in the cell, whereby an inhibition of the activity of the Mob-5 receptor indicates the agent has anti-cancer activity, thereby screening the agent for anti-cancer activity.

60. A method of inhibiting a cellular transformation phenotype induced by an oncogene whose product functions upstream of a mob-5 gene product, comprising inhibiting formation of an active Mob-5 receptor in a cell containing the upstream oncogene so as to inhibit the expression of the cellular transformation phenotype of the upstream oncogene.

61. The method of claim 60, wherein the cellular transformation phenotype is inhibited by a nucleic acid antisense to a Mob-5 receptor gene.

62. The method of claim 60, wherein the cellular transformation phenotype is induced by a ras oncogene product.

63. A method of inhibiting a cellular transformation phenotype induced by an oncogene whose product functions upstream of a mob-5 gene product, comprising inhibiting an active Mob-5 receptor in a cell containing the upstream oncogene so as to inhibit the expression of the cellular transformation phenotype of the upstream oncogene.

64. The method of claim 63, wherein the Mob-5 receptor is inhibited by an antibody to the Mob-5 receptor.

65. The method of claim 63, wherein the cellular transformation phenotype is induced by a ras oncogene product.

66. A method for inhibiting a mob-5 induced cellular transformation phenotype comprising inhibiting the formation of active Mob-5 receptor in a cell so as to inhibit the expression of the mob-5 induced cellular transformation phenotype.

67. The method of claim 66, wherein the inhibition of the formation of an active Mob-5 receptor is by a nucleic acid antisense to the Mob-5 receptor gene.

68. A method for inhibiting a mob-5 induced cellular transformation phenotype comprising inhibiting an active Mob-5 receptor in a cell so as to inhibit the expression of the mob-5 induced cellular transformation phenotype.

69. The method of claim 68, wherein the active Mob-5 receptor is inhibited by an antibody to the Mob-5 receptor.

70. The method of claim 68, wherein the active Mob-5 receptor is inhibited by administering to the cell an altered Mob-5 which binds to the Mob-5 receptor, whereby binding of the altered Mob-5 to the receptor inhibits the binding of Mob-5 to the receptor, thereby inhibiting the expression of the mob-5 induced cellular transformation phenotype.

71. A method of detecting the presence of cancer in a patient comprising:
a. contacting a sample from the patient with an antibody to a Mob-5 receptor;
b. detecting the binding of the antibody with an antigen in the sample, wherein binding of antigen to the antibody indicates the presence of Mob-5 receptor antigen in the sample and wherein Mob-5 receptor antigen in the sample indicates the presence of cancer in the patient, thereby detecting the presence of cancer in the patient.

72. The method of claim 71, wherein the cancer is a ras-induced cancer.

73. The method of claim 71, wherein the cancer is a colorectal, cancer.

74. A method of detecting the presence of cancer in a patient comprising:
a. contacting a sample from the patient with a Mob-5 receptor antigen;
b. detecting the binding of the antigen with an antibody in the sample, wherein binding of antigen to the antibody indicates the presence of Mob-5 receptor antibody in the sample and wherein Mob-5 receptor antibody in the sample indicates the presence of cancer in the patient, thereby detecting the presence of cancer in the patient.

75. The method of claim 74, wherein the cancer is a ras-induced cancer.

76. The method of claim 74, wherein the cancer is a colorectal, cancer.

77. A method of detecting the Mob-5 receptor comprising, contacting a cell with a Mob5-AP fusion protein, detecting the binding of the Mob5-AP fusion protein to the cell, wherein binding of the Mob5-AP to the cell indicates the cell is a Mob-5 receptor expressing cell, thus detecting the Mob-5 receptor.

78. A method of detecting the presence of cancer in a patient comprising:
a. contacting a sample from the patient with a mixture of biotinylated monoclonal antibody to Mob-5 and Streptavidin-horseradish peroxidase (HRP);
b. adding Gel Blue A Sepharose beads to the sample of a);
c. spinning down the beads;
d. removing supernatant;
e. adding HRP substrate
f. detecting the presence of color, wherein the presence of color indicates the presence of Mob-5 antigen in the sample and wherein Mob-5 antigen in the sample indicates the presence of cancer in the patient, thereby detecting the presence of cancer in the patient.

79. The method of claim 78, wherein the antibody to Mob-5 is the monoclonal antibody produced from hybridoma clone L-14.

80. The antibody of claim 21, wherein the antibody is a monoclonal antibody produced from hybridoma clone L-14.
